(19) United States
(12) Patent Application Publication
(10) Pub. No.: US 2006/0210974 A1
(43) Pub. Date: Sep. 21, 2006

(54) Compositions and methods for preventing, treating and diagnosing diabetes

(75) Inventors: Shonna Moodie, San Francisco, CA (US); Thomas A Gustafson, Danville, CA (US); Chi-Wai Wong, Hayward, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Metabolex, Inc., Hayward, CA (US)

(21) Appl. No.: 10/515,892

(22) PCT Filed: Jun. 13, 2003
(86) PCT No.: PCT/US03/18976

§ 371 Date: Oct. 28, 2005

Related U.S. Application Data
(60) Provisional application No. 60/388,716, filed on Jun. 13, 2002.

(51) Int. Cl. C12Q 1/68; G01N 33/567; A61K 39/395; A61K 48/00

(52) U.S. Cl. 435/6; 435/7.2; 514/44; 424/145.1

(57) ABSTRACT

The invention provides methods and compositions for preventing, treating and diagnosing diabetes.

### CROSS-REFERENCE TO RELATED APPLICATIONS

The present application claims benefit of priority to U.S. Provisional Patent Application No. 60/388716, filed Jun. 13, 2002, which is incorporated by reference for all purposes.

### FIELD OF THE INVENTION

This invention relates methods and compositions for preventing, treating and diagnosing diabetes.

### BACKGROUND OF THE INVENTION

Proteolysis is a ubiquitous mechanism that the cell employs to regulate the function and fate of proteins. Accordingly the number of proteases identified is large. There is a growing recognition of the function that proteases play in a wide range of physiological processes. Proteases have been found to play a role in defense mechanisms that protect against tissue damage and infection (e.g., proteolytic cascade of blood coagulation, fibrinolysis and complement system), to act as regulatory elements through the proteolytic activation of prohormones and zymogens, to induce the destruction of the extracellular matrix, to control tissue turnover and reorganization, and, to allow protein degradation in the lysosome.

Proteases are hydrolytic enzymes that share a general mechanism of catalytic cleavage of peptide bonds in protein and peptide substrates. Proteases are classified into four major groups: serine, cysteine, aspartate and metallo. Examples of serine proteases include trypsin, chymotrypsin, enterokinase, serum complement, and blood coagulation factors (Vindigni, Combinatorial & High Throughput Screening, 2139 (1999)).

Cysteine proteases can be grouped into two super families: the family of enzymes related to interleukin 1β converting enzyme (ICE) and the papain super family. ICE plays a role in inflammation and programmed cell death. Cysteine proteases of the papain family are mostly localized in the lysosomes comprising the cysteine cathepsins. These enzymes have largely been viewed as mediators for terminal digestion of endocytized and endogenous proteins entering the lysosomes (Chapman et al., Annu. Rev. Physiol., 59:63 (1997); McGrath, Annu. Rev. Biophys. Biolmol. Struct., 28:181 (1999); Buhling et al., In CELLULAR PEPTIDASES IN IMMUNE FUNCTION AND DISEASES (Eds., Ed Langner & Ansorge) pp. 241-254; Turk et al., EMBO. J., 20:4629 (2001)).

Aspartic proteases include cathepsin D, gastricin, pepsin and rennin. Connective tissue remodeling involves the breakdown and often the resynthesis of the extracellular matrix. Modification-of the ECM is an important component of many biological events including wound healing, angiogenesis, ovulation, embryogenesis and growth plate remodeling. The ECM is comprised of collagens, gelatins, elastics, proteoglycans, fibronectins, laminins and a variety of proteins, so that several proteases are required to degrade it. The most prominent group of such enzymes is the matrix metalloproteases (MMP) which include collagenases, gelatinases, stromelysins and matrilysins.

Besides being necessary from a physiological point of view, proteases are potentially hazardous to the surrounding cellular environment and their activity must therefore be precisely controlled by the respective cell or tissue. The control of proteases is normally achieved by regulation of expression, or secretion, or activation of pro-proteases, by degradation of mature enzymes and by the inhibition of their proteolytic activity. Protease inhibitors adopt many different structures, ranging in size from small to large macromolecular structures much larger than their target enzyme. Except for α2-macroglobulin, which has very broad specificity, the protease inhibitors are very specific for the type of protease they inhibit.

At least four distinct families of serine protease inhibitors are known in mammals: the serpins, the Kazal, the Kunitz and the leuko-protease type (Otlewski et al., Acta Biochem. Polonica, 46:531 (1999)). Many serpins are serum proteins with specificities directed toward serine proteases, whose catalytic activities control processes such as blood coagulation and fibrinolysis.

Kunitz-type protease inhibitors are usually low molecular weight proteins with one or more inhibitory domains and may be particularly important where there is a need to control a cascade of proteolytic reactions as in blood clotting.

The Kazal inhibitors function, much like the Kunitz-type family, with their structure maintained by three disulfide bonds. The most widely studied Kazal family members are the ovamucoids, avian egg white protease inhibitors that contain up to six Kazal type domains and inhibit trypsin, chymotrypsin, elastase and subtilisin.

The leukoprotease inhibitors are generally of low molecular weight and consist of two inhibitory domains each containing four conserved disulphides. The leukoprotease inhibitors almost certainly serve to control the activities of proteases that might damage mucosal surfaces, including elastases released from neutrophils in the lung.

The inhibitors of cysteine proteases can be largely grouped into two families: cystatins and kininogens. The cystatin super family can be grouped into two distinct families. The first family, stefins, are proteins that lack disulfide bonds and carbohydrate. Stefins include cystatin A and cystatin B. The second family possess two disulfide bonds and includes cystatin C, cystatin D, cystatin S, cystatin SN, cystatin SA and sarcocystatin A.

Kininogens are plasma glycoproteins and have up to nine disulphide bonds. Cystatins inhibit activities of cysteine proteases such as plant enzymes papain, papaya protease III and the mammalian enzymes dipeptidyl peptidases I (cathepsin C), and the lysosomal cathepsins B, H, and L. The major mechanism that leads to the inactivation of the MMP is through binding to two classes of inhibitor, α2-macroglobulin or to the tissue inhibitor of metalloproteases (TIMP) (Roberts et al., Critical Rev. Euk. Gene Exp., 5:385 (1995)).

All known protease inhibitors prevent access of substrates to the protease catalytic center through steric hindrance. Domains that mediate this interaction between the protease inhibitor and the target protease have been described. In most cases all members of a specific inhibitor family are directed against the same class of target protease. Only a few protease inhibitors exhibit dual activity simultaneously exerted towards proteases from distinct classes. One example is the KAZAL domain which is found to bind in a canonical substrate-like manner to the active sites of serine proteases. The prototype KAZAL inhibitor is pancreatic secretory trypsin inhibitor that appears to be present in all animals. The KAZAL inhibitors function with their structure maintained by three disulphide bonds. The thyroglobulin-1 repeats are found in a variety of proteins of different function. The repeats are thought to be involved in the control of proteolytic degradation (Guncar et al., EMBO J., 18, 793-803 (1999)). The domain usually contains six conserved cysteines. These form three disulphide bridges. Thyroglobulin-1 repeat domains are found within the IGFBPs which represents a large family of proteins that inhibit the growth stimulating effects of the IGFs by inhibiting the degradation of IGFBP-4 through binding to and inhibiting the protease (Fowlkes et al, Endocrinol., 138:2280 (1997)). Kunitz-type inhibitory domains are characterized by the conserved placement of six cysteine residues which form three disulfide bonds.

Diabetes mellitus can be divided into two clinical syndromes, Type 1 and Type 2 diabetes mellitus. Type 1, or insulin-dependent diabetes mellitus (IDDM), is a chronic autoimmune disease characterized by the extensive loss of beta cells in the pancreatic Islets of Langerhans, which produce insulin. As these cells are progressively destroyed, the amount of secreted insulin decreases, eventually leading to hyperglycemia (abnormally high level of glucose in the blood) when the amount of secreted insulin drops below the level required for euglycemia (normal blood glucose level). Although the exact trigger for this immune response is not known, patients with IDDM have high levels of antibodies against proteins expressed in pancreatic beta cells. However, not all patients with high levels of these antibodies develop IDDM.

Type 2 diabetes (also referred to as non-insulin dependent diabetes mellitus (NIDDM)) develops when muscle, fat and liver cells fail to respond normally to insulin. This failure to respond (called insulin resistance) may be due to reduced numbers of insulin receptors on these cells, or a dysfunction of signaling pathways within the cells, or both. The beta cells initially compensate for-this insulin resistance by increasing insulin output. Over time, these cells become unable to produce enough insulin to maintain normal glucose levels, indicating progression to Type 2 diabetes.

Type 2 diabetes is brought on by a combination of genetic and acquired risk factors—including a high-fat diet, lack of exercise, and aging. Worldwide, Type 2 diabetes has become an epidemic, driven by increases in obesity and a sedentary lifestyle, widespread adoption of western dietary habits, and the general aging of the population in many countries. In 1985, an estimated 30 million people worldwide had diabetes—by 2000, this figure had increased 5-fold, to an estimated 154 million people. The number of people with diabetes is expected to double between now and 2025, to about 300 million.

Type 2 diabetes is a complex disease characterized by defects in glucose and lipid metabolism. Typically there are perturbations in many metabolic parameters including increases in fasting plasma glucose levels, free fatty acid levels and triglyceride levels, as well as a decrease in the ratio of HDL/LDL. As discussed above, one of the principal underlying causes of diabetes is thought to be an increase in insulin resistance in peripheral tissues, principally muscle and fat. The present invention addresses this and other problems.

### BRIEF SUMMARY OF THE INVENTION

This invention provides compositions and methods related to the Smooth Muscle Associated Protein-2 (SMAP-2). For example, the present invention provides methods for identifying an agent for treating a diabetic or pre-diabetic individual. In some embodiments, the methods comprise the steps of: (i) contacting an agent with a solution comprising a polypeptide encoded by a nucleic acid that hybridizes under stringent conditions to a nucleic acid encoding SEQ ID NO:2, SEQ ID NO:4, SEQ ID NO:6 or SEQ ID NO:9; (ii) selecting an agent that decreases the expression or activity of the polypeptide or that binds to the polypeptide; and (iii) testing the selected agent for the ability to modulate insulin sensitivity in a cell, thereby identifying an agent capable of modulating insulin sensitivity in a cell.

In some embodiments, the methods comprise selecting an agent that decreases expression of the polypeptide. In some embodiments, the methods comprise selecting an agent that decreases activity of the polypeptide. In some embodiments, the methods comprise selecting an agent that binds to the polypeptide.

In some embodiments, the contacting step is performed in vitro. In some embodiments, the testing step comprises administering the agent to an animal and testing the animal for modulated insulin sensitivity. In some embodiments, the animal exhibits insulin resistance prior to administration.

In some embodiments, the testing step comprises contacting a cell expressing the polypeptide with the agent and testing the cell for modulated insulin sensitivity. In some embodiments, the testing comprises selecting an agent that increases glucose uptake in the cell compared to a cell not contacted with the agent. In some embodiments, the testing comprises selecting an agent that increases GLUT4 translocation in the cell compared to a cell not contacted with the agent.

In some embodiments, the cell is an adipocyte, heart cell or skeletal muscle cell. In some embodiments, the cell is a human cell.

In some embodiments, the amino acid sequence comprises SEQ ID NO:2. In some embodiments, the amino acid sequence comprises SEQ ID NO:4. In some embodiments, the amino acid sequence comprises SEQ ID NO:6. In some embodiments, the amino acid sequence comprises SEQ ID NO:9.

In some embodiments, the agent is an antibody. In some embodiments, the antibody is a monoclonal antibody. In some embodiments, the agent is an antisense polynucleotide.

In some embodiments, the testing step comprises testing glucose uptake of the cell. In some embodiments, the testing step comprises testing GLUT4 translocation in the cell.

The present invention also provides methods of treating a diabetic or pre-diabetic animal. In some embodiments, the methods comprise administering to the animal a therapeutically effective amount of an agent identified by (i) contacting an agent with a solution comprising a polypeptide encoded by a nucleic acid that hybridizes under stringent conditions to a nucleic acid encoding SEQ ID NO:2; (ii) selecting an agent that decreases the expression or activity of the polypeptide or that binds to the polypeptide; and (iii) testing the selected agent for the ability to modulate insulin sensitivity in a cell.

In some embodiments, the agent is an antibody. In some embodiments, the antibody is a monoclonal antibody. In some embodiments, the agent is an antisense polynucleotide. In some embodiments, animal is a human.

In some embodiments, the methods comprise administering to the animal a therapeutically effective amount of an agent that specifically binds to SEQ ID NO:2. In some embodiments, the agent is a polypeptide. In some embodiments, the agent is an antibody. In some embodiments, the antibody is a monoclonal antibody. In some embodiments, the animal is a human. In some embodiments, the agent is an antisense polynucleotide.

The present invention also provides methods of diagnosing a diabetic or pre-diabetic individual. In some embodiments, the methods comprise detecting in a sample from the individual the level of a polypeptide encoded by a nucleic acid that hybridizes under stringent conditions to a nucleic acid encoding SEQ ID NO:2, wherein an increased level of the polypeptide in the sample compared to a level of the polypeptide in a lean person or a previous sample from the individual indicates that the individual is diabetic or pre-diabetic.

In some embodiments, the individual has Type 2 diabetes. In some embodiments, the individual is pre-diabetic. In some embodiments, the sample is a blood, urine or tissue sample.

In some embodiments, the detecting step comprises contacting the sample with an antibody that specifically binds to the polypeptide.

In some embodiments, the methods comprise detecting in a sample from the individual the level of a polynucleotide that hybridizes under stringent conditions to a nucleic acid encoding SEQ ID NO:2, wherein an increased level of the polynucleotide in the sample compared to a level of the polynucleotide in a lean person or a previous sample from the individual indicates that the individual is diabetic or pre-diabetic. In some embodiments, the detecting step comprises quantifying mRNA that hybridizes under stringent conditions to a nucleic acid encoding SEQ ID NO:2. In some embodiments, the mRNA is reverse transcribed and amplified in a polymerase chain reaction. In some embodiments, the sample is a blood, urine or tissue sample.

The present invention also provides isolated nucleic acids comprising a polynucleotide encoding a polypeptide comprising one or two EF calcium-binding hands (amino acids 351 to 379 and 388 to 416 of SEQ ID NO:2), a Kazal domain (amino acids 40 to 84 of SEQ ID NO:2) and/or two thyroglobulin-1 repeat domains (amino acids 90 to 153 and 216 to 281 of SEQ ID NO:2). In some embodiments, the polypeptide is as displayed in SEQ ID NO:2. In some embodiments, the polynucleotide is SEQ ID NO:1.

The present invention also provides an expression cassette comprising a heterologous promoter operably linked to a nucleic acid comprising a polynucleotide encoding a polypeptide as displayed in SEQ ID NO:2.

The present invention also provides host cells transfected with a nucleic acid comprising a polynucleotide encoding a polypeptide as displayed in SEQ ID NO:2. In some embodiments, the host cell is a human cell. In some embodiments, the host cell is a bacteria.

The present invention also provides isolated polypeptides comprising one or two EF calcium-binding hands (amino acids 351 to 379 and 388 to 416 of SEQ ID NO:2), a Kazal domain (amino acids 40 to 84 of SEQ ID NO:2) and/or two thyroglobulin-1 repeat domains (amino acids 90 to 153 and 216 to 281 of SEQ ID NO:2). In some embodiments, the polypeptide comprises SEQ ID NO:2.

The present invention also provides an expression cassette comprising a promoter operably linked to a heterologous polynucleotide, wherein the promoter comprises SEQ ID NO:10 or SEQ ID NO:11. In some embodiments, the expression cassette comprises a polynucleotide encoding a polypeptide. In some embodiments, the polypeptide is a reporter gene product.

The present invention also provides cells comprising an expression cassette comprising a promoter operably linked to a heterologous polynucleotide, wherein the promoter comprises SEQ ID NO:10 or SEQ ID NO:11.

The present invention also provides methods of expressing a polynucleotide in a muscle cell. In some embodiments, the methods comprise introducing an expression cassette comprising a promoter operably linked to a heterologous polynucleotide, wherein the promoter comprises SEQ ID NO:10 or SEQ ED NO:11.

### DEFINITIONS

“Insulin sensitivity” refers to effect of insulin on glucose uptake in a cell. Sensitivity can be determined at an organismal, tissue or cellular level. For example, blood or urine glucose levels following a glucose tolerance test are indicative of insulin sensitivity. Other methods of measuring insulin sensitivity include, e.g., measuring glucose uptake (see, e.g., Garcia de Herreros, A., and Birnbaum, M. J. J. Biol. Chem. 264, 19994-19999 (1989); Klip, A., Li, G., and Logan, W. J. Am. J. Physiol. 247, E291-296 (1984)), measuring the glucose infusion rate (GINF) into tissue such as the skeletal muscle (see, e.g., Ludvik et al., J. Clin. Invest. 100:2354 (1997); Frias et al., Diabetes Care 23:64, (2000)) and measuring sensitivity of GLUT4 translocation (e.g., as described herein) in response to insulin.

“Predisposition for diabetes” occurs in a person when the person is at high risk for developing diabetes. A number of risk factors are known to those of skill in the art and include: genetic factors (e.g., carrying alleles that result in a higher occurrence of diabetes than in the average population or having parents or siblings with diabetes); overweight (e.g., body mass index (BMI) greater or equal to 25 kg/m²); habitual physical inactivity, race/ethnicity (e.g., African-American, Hispanic-American, Native Americans, Asian-Americans, Pacific Islanders); previously identified impaired fasting glucose or impaired glucose tolerance, hypertension (e.g., greater or equal to 140/90 mmHg in adults); HDL cholesterol greater or equal to 35 mg/dl; triglyceride levels greater or equal to 250 mg/dl; a history of gestational diabetes or delivery of a baby over nine pounds; and/or polycystic ovary syndrome. See, e.g., “Report of the Expert Committee on the Diagnosis and Classification of Diabetes Mellitus” and “Screening for Diabetes” Diabetes Care 25(1): S5-S24 (2002).

A “lean individual,” when used to compare with a sample from a patient, refers to an adult with a fasting blood glucose level less than 110 mg/dl or a 2 hour PG reading of 140 mg/dl. “Fasting” refers to no caloric intake for at least 8 hours. A “2 hour PG” refers to the level of blood glucose after challenging a patient to a glucose load containing the equivalent of 75 g anhydrous glucose dissolved in water. The overall test is generally referred to as an oral glucose tolerance test (OGTT). See, e.g., Diabetes Care, Supplement 2002, American Diabetes Association: Clinical Practice Recommendations 2002. The level of a polypeptide in a lean individual can be a reading from a single individual, but is typically a statistically relevant average from a group of lean individuals. The level of a polypeptide in a lean individual can be represented by a value, for example in a computer program.

A “pre-diabetic individual,” when used to compare with a sample from a patient, refers to an adult with a fasting blood glucose level greater than 110 mg/dl but less than 126 mg/dl or a 2 hour PG reading of greater than 140 mg/dl but less than 200 mg/dl. A “diabetic individual,” when used to compare with a sample from a patient, refers to an adult with a fasting blood glucose level greater than 126 mg/dl or a 2 hour PG reading of greater than 200 mg/dl.

A “SMAP-2 nucleic acid” or “SMAP-2 polynucleotide” of the invention is a subsequence or full-length polynucleotide sequence of a gene that encodes a SMAP-2 polypeptide. Exemplary SMAP-2 nucleic acids of the invention include sequences substantially identical to SEQ ID NOs: 1, 3, 5, 7, and 8. Several nucleotide sequences encoding human SMAP-2 are deposited in Genbank (e.g., Accession numbers AB014737 and AB014730). The nucleotide sequence encoding the mouse ortholog of SMAP-2 (designated Secreted Modular Calcium-Binding Protein-2 (SMOC-2) is deposited in Genbank under the Accession number of AJ249901.

A nucleotide sequence encoding human SMAP-2 is deposited in Genbank under the Accession number AB014730 and has an open reading frame beginning at position 21 and ending with a stop codon at position 1361. See also, SEQ ID NO:3. Nucleotides 21 to 83 of AB014730 encode the signal peptide, nucleotides 138 to 272 encode the Kazal domain, nucleotides 288 to 479 and 666 to 863 encode the two thyroglobulin-1 repeat domains and nucleotides 1071 to 1157 and 1182 to 1268 encodes the two EF-hand domains.

“SMAP-2 polypeptide” or “SMAP-2” refers to a polypeptide, or fragment thereof, that is a protease inhibitor and is substantially identical to SEQ ID NO:2, SEQ ID NO:4, SEQ ID NO:6, and SEQ ID NO:9, as well as the polypeptide encoded by SEQ ID NO:7. Components of SMAP-2 sequence can include, e.g., an export signal sequence (amino acids 1 to 21 of SEQ ID NO:2), one or two EF calcium-binding hands (amino acids 351 to 379 and 388 to 416 of SEQ ID NO:2) (Kawasaki, H & Kretsinger R. H. Protein Profile, 2, 297-490 (1995); Kawasaki et al., Biometals 11, 277-295 (1998)), a Kazal domain (amino acids 40 to 84 of SEQ ID NO:2) (Lu et al., Proc. Natl. Acad. Sci. USA. 98, 1410-1415 (2001)) and two thyroglobulin-1 repeat domains (amino acids 90 to 153 and 216 to 281 of SEQ ID NO:2) (Molina et al., Eur. J. Biochem., 240:125-133, (1996); Fowlkes et al., Endocrinol., 138:2280-2285 (1997)). A consensus sequence of thyroglobulin-1 repeat domains has been derived from sequence similarity searches. Thyroglobulin-1 repeat domains has been found to contain a central core of conserved residues including two highly conserved motifs, QC and CWCV (SEQ ID NO:12) (Molina et al., Eur. J. Biochem. 240:125-133 (1996)).

“SMAP-2 activity” refers to the ability of a SMAP-2 polypeptide to bind to a second polypeptide. In some cases, the second protein is a protease, and binding of SMAP-2 to the protease inhibits activity of the protease. Protease activity, and therefore inhibition of protease activity, can be measured by determining the amount of protease substrate cleaved over time in the presence of the protease. Inhibition can be measured by determining the proteolytic activity of the protease in the presence and absence of SMAP-2. However, SMAP-2 activity is not limited to protease inhibition. SMAP-2 activity can include other types of protein-protein interactions that regulate signal transduction. See, e.g., Hoegy et al., J. Biol. Chem. 276(5):3203-3214 (2001), describing a protease inhibitor that modulates signal transduction by a method other than protease inhibition.

SMAP-2 activity can also be measured indirectly by monitoring a cell's insulin sensitivity in the presence or absence of SMAP-2. For example, activity of SMAP-2 can be analyzed by expressing SMAP-2 in a cell such as an adipocyte and monitoring GLUT4 translocation and/or glucose uptake. Changes in GLUT4 translocation in response to treatment with such molecules or other SMAP-2 modulators provide a measure of changes in SMAP-2 activity. Relative SMAP-2 activity is proportional to inhibition of GLUT4 translocation and/or glucose uptake in adipocytes.

An “agonist of SMAP-2” refers to an agent that binds to SMAP-2, stimulates, increases, activates, facilitates, enhances activity or expression of SMAP-2.

An “antagonist of SMAP-2” refers to an agent that binds to, partially or totally blocks stimulation, decreases, prevents, delays activation, inactivates, desensitizes, or down regulates the activity or expression of SMAP-2.

“Antibody” refers to a polypeptide substantially encoded by an immunoglobulin gene or immunoglobulin genes, or fragments thereof which specifically bind and recognize an analyte (antigen). The recognized immunoglobulin genes include the kappa, lambda, alpha, gamma, delta, epsilon and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

An exemplary immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (V_{L}) and variable heavy chain (V_{H}) refer to these light and heavy chains respectively.

Antibodies exist, e.g., as intact immunoglobulins or as a number of well characterized fragments produced by digestion with various peptidases. Thus, for example, pepsin digests an antibody below the disulfide linkages in the hinge region to produce F(ab)′₂, a dimer of Fab which itself is a light chain joined to V_{H}-C_{H}1 by a disulfide bond. The F(ab)′₂ may be reduced under mild conditions to break the disulfide linkage in the hinge region, thereby converting the F(ab)′₂ dimer into a Fab′ monomer. The Fab′ monomer is essentially an Fab with part of the hinge region (see, Paul (Ed.) Fundamental Immunology, Third Edition, Raven Press, NY (1993)). While various antibody fragments are defined in terms of the digestion of an intact antibody, one of skill will appreciate that such fragments may be synthesized de novo either chemically or by utilizing recombinant DNA methodology. Thus, the term antibody, as used herein, also includes antibody fragments either produced by the modification of whole antibodies or those synthesized de novo using recombinant DNA methodologies (e.g., single chain Fv).

The terms “peptidomimetic” and “mimetic” refer to a synthetic chemical compound that has substantially the same structural and functional characteristics of the SMAP-2 antagonists or agonists of the invention. Peptide analogs are commonly used in the pharmaceutical industry as non-peptide drugs with properties analogous to those of the template peptide. These types of non-peptide compound are termed “peptide mimetics” or “peptidomimetics” (Fauchere, J. Adv. Drug Res. 15:29 (1986); Veber and Freidinger TINS p. 392 (1985); and Evans et al. J. Med. Chem. 30:1229 (1987), which are incorporated herein by reference). Peptide mimetics that are structurally similar to therapeutically useful peptides may be used to produce an equivalent or enhanced therapeutic or prophylactic effect. Generally, peptidomimetics are structurally similar to a paradigm polypeptide (i.e., a polypeptide that has a biological or pharmacological activity), such as an SMAP-2 polypeptide antagonist, but have one or more peptide linkages optionally replaced by a linkage selected from the group consisting of, e.g., —CH2NH—, —CH2S—, —CH2-CH2-, —CH═CH— (cis and trans), —COCH2-, —CH(OH)CH2-, and —CH2SO—. The mimetic can be either entirely composed of synthetic, non-natural analogues of amino acids, or, is a chimeric molecule of partly natural peptide amino acids and partly non-natural analogs of amino acids. The mimetic can also incorporate any amount of natural amino acid conservative substitutions as long as such substitutions also do not-substantially alter the mimetic's structure and/or activity. For example, a mimetic composition is within the scope of the invention if it is capable of carrying out the binding or other activities of SMAP-2, or a SMAP-2 agonist or antagonist.

The term “gene” means the segment of DNA involved in producing a polypeptide chain; it includes regions preceding and following the coding region (leader and trailer) as well as intervening sequences (introns) between individual coding segments (exons).

The term “isolated,” when applied to a nucleic acid or protein, denotes that the nucleic acid or protein is essentially free of other cellular components with which it is associated in the natural state. It is preferably in a homogeneous state although it can be in either a dry or aqueous solution. Purity and homogeneity are typically determined using analytical chemistry techniques such as polyacrylamide gel electrophoresis or high performance liquid chromatography. A protein that is the predominant species present in a preparation is substantially purified. In particular, an isolated gene is separated from open reading frames that flank the gene and encode a protein other than the gene of interest. The term “purified” denotes that a nucleic acid or protein gives rise to essentially one band in an electrophoretic gel. Particularly, it means that the nucleic acid or protein is at least 85% pure, more preferably at least 95% pure, and most preferably at least 99% pure.

As used herein, the terms “nucleic acid” and “polynucleotide” are used interchangeably. Use of the term “polynucleotide” includes oligonucleotides (i.e., short polynucleotides). This term also refers to deoxyribonucleotides, ribonucleotides, and naturally occurring variants, and can also refer to synthetic and/or non-naturally occurring nucleic acids (i.e., comprising nucleic acid analogues or modified backbone residues or linkages), such as, for example and without limitation, phosphorothioates, phosphoramidates, methyl phosphonates, chiral-methyl phosphonates, 2-O-methyl ribonucleotides, peptide-nucleic acids (PNAs), and the like. Unless otherwise indicated, a particular nucleic acid sequence also implicitly encompasses conservatively modified variants thereof (e.g., degenerate codon substitutions) and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Cassol et al. (1992); Rossolini et al., Mol. Cell. Probes 8:91-98 (1994)). The term nucleic acid is used interchangeably with gene, cDNA, and mRNA encoded by a gene.

The terms “polypeptide,” “peptide” and “protein” are used interchangeably herein to refer to a polymer of amino acid residues. The terms apply to amino Acid polymers in which one or more amino acid residue is an artificial chemical mimetic of a corresponding naturally occurring amino acid, as well as to naturally occurring amino acid polymers and non-naturally occurring amino acid polymers. As used herein, the terms encompass amino acid chains of any length, including full-length proteins (i.e., antigens), wherein the amino acid residues are linked by covalent peptide bonds.

The term “amino acid” refers to naturally occurring and synthetic amino acids, as well as amino acid analogs arid amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. “Amino acid mimetics” refers to chemical compounds that have a structure that is different from the general chemical structure of an amino acid, but which functions in a manner similar to a naturally occurring amino acid.

Amino acids may be referred to herein by either the commonly known three letter symbols or by the one-letter symbols recommended by the IUPAC-IUB Biochemical Nomenclature Commission. Nucleotides, likewise, may be referred to by their commonly accepted single-letter codes.

“Conservatively modified variants” applies to both amino acid and nucleic acid sequences. With respect to particular nucleic acid sequences, “conservatively modified variants” refers to those nucleic acids that encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein that encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid that encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

The following eight groups each contain amino acids that are conservative substitutions for one another:
1) Alanine (A), Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine (S), Threonine (T); and
8) Cysteine (C), Methionine (M)
   (see, e.g., Creighton, Proteins (1984)).

“Percentage of sequence identity” is determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The terms “identical” or percent “identity,” in the context of two or more nucleic acids or polypeptide sequences, refer to two or more sequences or subsequences that are the same or have a specified percentage of amino acid residues or nucleotides that are the same (i.e., 60% identity, optionally 65%, 70%, 75%, 80%, 85%, 90%, 95%, 98%, 99% identity over a specified region, or, when not specified, over the entire sequence), when compared and aligned for maximum correspondence over a comparison window, or designated region as measured using one of the following sequence comparison algorithms or by manual alignment and visual inspection. Such sequences are then said to be “substantially identical.” This definition also refers to the complement of a test sequence. Similarly, “substantially complementary” polynucleotides are at least 60% complementary, optionally 65%, 70%, 75%, 80%, 85%, 90%, 95%, 98%, 99% complementary over a specified region, or, when not specified, over the entire sequence. Optionally, the identity or complementarity exists over a region that is at least about 50 nucleotides in length, or more preferably over a region that is 100 to 500 or 1000 or more nucleotides in length.

The term “similarity,” or percent “similarity,” in the context of two or more polypeptide sequences, refer to two or more sequences or subsequences that have a specified percentage of amino acid residues that are either the same or similar as defined in the 8 conservative amino acid substitutions defined above (i.e., 60%, optionally 65%, 70%, 75%, 80%, 85%, 90%, 95%, 98% or 99% similar over a specified region or, when not specified, over the entire sequence), when compared and aligned for maximum correspondence over a comparison window, or designated region as measured using one of the following sequence comparison algorithms or by manual alignment and visual inspection. Such sequences are then said to be “substantially similar.” Optionally, this identity exists over a region that is at least about 50 amino acids in length, or more preferably over a region that is at least about 100, 200, 300, 400, 500 or 1000 or more amino acids in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well known in the art. Optimal alignment of sequences for comparison can be conducted, e.g., by the local homology algorithm of Smith and Waterman (1970) Adv. Appl. Math. 2:482c, by the homology alignment algorithm of Needleman and Wunsch (1970) J. Mol. Biol. 48:443, by the search for similarity method of Pearson and Lipman (1988) Proc. Nat'l. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Ausubel et al., Current Protocols in Molecular Biology (1995 supplement)).

One example of a useful algorithm is PILEUP. PILEUP creates a multiple sequence alignment from a group of related sequences using progressive, pairwise alignments to show relationship and percent sequence identity. It also plots a tree or dendogram showing the clustering relationships used to create the alignment. PILEUP uses a simplification of the progressive alignment method of Feng and Doolittle (1987) J. Mol. Evol. 35:351-360. The method used is similar to the method described by Higgins and Sharp (1989) CABIOS 5:151-153. The program can align up to 300 sequences, each of a maximum length of 5,000 nucleotides or amino acids. The multiple alignment procedure begins with the pairwise alignment of the two most similar sequences, producing a cluster of two aligned sequences. This cluster is then aligned to the next most related sequence or cluster of aligned sequences. Two clusters of sequences are aligned by a simple extension of the pairwise alignment of two individual sequences. The final alignment is achieved by a series of progressive, pairwise alignments. The program is run by designating specific sequences and their amino acid or nucleotide coordinates for regions of sequence comparison and by designating the program parameters. Using PILEUP, a reference sequence is compared to other test sequences to determine the percent sequence identity relationship using the following parameters: default gap weight (3.00), default gap length weight (0.10), and weighted end gaps. PILEUP can be obtained from the GCG sequence analysis software package, e.g., version 7.0 (Devereaux et al. (1984) Nuc. Acids Res. 12:387-395).

Another example of an algorithm that is suitable for determining percent sequence-identity and sequence similarity are the BLAST and BLAST 2.0 algorithms, which are described in Altschul et al. (1977) Nuc. Acids Res. 25:3389-3402, and Altschul et al. (1990) J. Mol. Biol. 215:403-410, respectively. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) or 10, M=5, N=−4 and a comparison of both strands. For amino acid sequences, the BLASTP program uses as defaults a wordlength of 3, and expectation (E) of 10, and the BLOSUM62 scoring matrix (see Henikoff and Henikoff(1989) Proc. Natl. Acad. Sci. USA 89:10915) alignments (B) of 50, expectation (E) of 10, M=5, N=−4, and a comparison of both strands.

The BLAST algorithm also performs a statistical analysis of the similarity between two sequences (see, e.g., Karlin and Altschul (1993) Proc. Natl. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

An indication that two nucleic acid sequences or polypeptides are substantially identical is that the polypeptide encoded by the first nucleic acid is immunologically cross reactive with the antibodies raised against the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequence.

The phrase “selectively (or specifically) hybridizes to” refers to the binding, duplexing, or hybridizing of a molecule only to a particular nucleotide sequence under stringent hybridization conditions when that sequence is present in a complex mixture (e.g., total cellular or library DNA or RNA).

The phrase “stringent hybridization conditions” refers to conditions under which a probe will hybridize to its target subsequence, typically in a complex mixture of nucleic acid, but to no other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Probes, “Overview of principles of hybridization and the strategy of nucleic acid assays” (1993). Generally, stringent conditions are selected to be about 5-10° C. lower than the thermal melting point (Tₘ) for the specific sequence at a defined ionic strength pH. The Tₘ is the temperature (under defined ionic strength, pH, and nucleic concentration) at which 50% of the probes complementary to the target hybridize to the target sequence at equilibrium (as the target sequences are present in excess, at Tₘ, 50% of the probes are occupied at equilibrium). Stringent conditions will be those in which the salt concentration is less than about 1.0 M sodium ion, typically about 0.01 to 1.0 M sodium ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. For selective or specific hybridization, a positive signal is at least two times background, optionally 10 times background hybridization. Exemplary stringent hybridization conditions can be as following: 50% formamide, 5×SSC, and 1% SDS, incubating at 42° C., or 5×SSC, 1% SDS, incubating at 65° C., with wash in 0.2×SSC, and 0.1% SDS at 55° C., 60° C., or 65° C. Such washes can be performed for 5, 15, 30, 60, 120, or more minutes.

Nucleic acids that do not hybridize to each other under stringent conditions are stilt substantially identical if the polypeptides that they encode are substantially identical. This occurs, for example, when a copy of a nucleic acid is created using the maximum codon degeneracy permitted by the genetic code. In such cases, the nucleic acids typically hybridize under moderately stringent hybridization conditions. Exemplary “moderately stringent hybridization conditions” include a hybridization in a buffer of 40% formamide, 1 M NaCl, 1% SDS at 37° C., and a wash in 1×SSC at 45° C. Such washes can be performed for 5, 15, 30, 60, 120, or more minutes. A positive hybridization is at least twice background. Those of ordinary skill will readily recognize that alternative hybridization and wash conditions can be utilized to provide-conditions of similar stringency.

The phrase “a nucleic acid sequence encoding” refers to a nucleic acid which contains sequence information for a structural RNA such as rRNA, a tRNA, or the primary amino acid sequence of a specific protein or peptide, or a binding site for a trans-acting regulatory agent. This phrase specifically encompasses degenerate codons (i.e., different codons which encode a single amino acid) of the native sequence or sequences that may be introduced to conform with codon preference in a specific host cell.

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (nonrecombinant) form of the cell or express native genes that are otherwise abnormally expressed, under-expressed or not expressed at all.

The term “heterologous” when used with reference to portions of a nucleic acid indicates that the nucleic acid comprises two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source. Similarly, a heterologous protein indicates that the protein comprises two or more subsequences that are not found in the same relationship to each other in nature (e.g., a fusion protein).

An “expression vector” is a nucleic acid construct, generated recombinantly or synthetically, with a series of specified nucleic acid elements that permit transcription of a particular nucleic acid in a host cell. The expression vector can be part of a plasmid, virus, or nucleic acid fragment. Typically, the expression vector includes a nucleic acid to be transcribed operably linked to a promoter.

The phrase “specifically (or selectively) binds to an antibody” or “specifically (or selectively) immunoreactive with”, when referring to a protein or peptide, refers to a binding reaction which is determinative of the presence of the protein in the presence of a heterogeneous population of proteins and other biologics. Thus, under designated immunoassay conditions, the specified antibodies bind to a particular protein and do not bind in a significant amount to other proteins present in the sample. Specific binding to an antibody under such conditions may require an antibody that is selected for its specificity for a particular protein. For example, antibodies raised against a protein having an amino acid sequence encoded by any of the polynucleotides of the invention can be selected to obtain antibodies specifically immunoreactive with that protein and not with other proteins, except for polymorphic variants. A variety of immunoassay formats may be used to select antibodies specifically immunoreactive with a particular protein. For example, solid-phase ELISA immunoassays, Western blots, or immunohistochemistry are routinely used to select monoclonal antibodies specifically immunoreactive with a protein. See, Harlow and Lane Antibodies, A Laboratory Manual, Cold Spring Harbor Publications, NY (1988) for a description of immunoassay formats and conditions that can be used to determine specific immunoreactivity. Typically, a specific or selective reaction will be at least twice the background signal or noise and more typically more than 10 to 100 times background.

“Inhibitors,” “activators,” and “modulators” of SMAP-2 expression or of SMAP-2 activity are used to refer to inhibitory, activating, or modulating molecules, respectively, identified using in vitro and in vivo assays for SMAP-2 expression or SMAP-2 activity, e.g., ligands, agonists, antagonists, and their homologs and mimetics. The term “modulator” includes inhibitors and activators. Inhibitors are agents that, e.g., inhibit expression of SMAP-2 or bind to, partially or totally block stimulation or protease inhibitor activity, decrease, prevent, delay activation, inactivate, desensitize, or down regulate the activity of SMAP-2, e.g., antagonists. Activators are agents that, e.g., induce or activate the expression of a SMAP-2 or bind to, stimulate, increase, open, activate, facilitate, enhance activation or protease inhibitor activity, sensitize or up regulate the activity of SMAP-2, e.g., agonists. Modulators include naturally occurring and synthetic ligands, antagonists and agonists (e.g., small chemical molecules, antibodies and the like that function as either agonists or antagonists). Such assays for inhibitors and activators include, e.g., applying putative modulator compounds to cells expressing SMAP-2 and then determining the functional effects on SMAP-2 activity, as described above. Samples or assays comprising SMAP-2 that are treated with a potential activator, inhibitor, or modulator are compared to control samples without the inhibitor, activator, or modulator to examine the extent of effect. Control samples (untreated with modulators) are assigned a relative SMAP-2 activity value of 100%. Inhibition of SMAP-2 is achieved when the SMAP-2 activity value relative to the control is about 80%, optionally 50% or 25, 10%, 5% or 1%. Activation of SMAP-2 is achieved when the SMAP-2 activity value relative to the control is 110%, optionally 150%, optionally 200, 300%, 400%, 500%, or 1000-3000% or more higher.

### BRIEF DESCRIPTION OF THE DRAWINGS

FIG. 1 is a bar graph displaying the expression levels of SMAP-2 in a variety of human tissues.

FIG. 2 illustrates a commercially available human multiple tissue northern blot hybridized with a SMAP-2 probe.

FIG. 3 is a bar graph displaying the upregulation of SMAP-2 gene expression in human diabetic skeletal muscle.

FIG. 4 is a bar graph displaying expression of SMAP-2 as determined by quantitative PCR in human skeletal muscle.

FIG. 5 is a bar graph displaying the upregulation of SMOC-2 gene expression in mouse db/db skeletal muscle.

FIG. 6 is a western blot displaying the secretion of SMAP-2 from adipocytes. Standard molecular weight markers (KDa) are shown on the left of FIG. 6. The “++” label indicates an aliquot of recombinant SMAP-2 used as a positive control on the immunoblot.

FIG. 7 is a bar graph demonstrating that overexpression of SMAP-2 inhibits insulin stimulated GLUT4 translocation in mouse adipocytes.

FIG. 8 is a bar graph illustrating glucose uptake in cells infected with adenovirus expressing either a GFP control (eGFP) or SMAP-2 at 100 or 200 MOI.

FIG. 9 illustrates the amino acid alignment between human and mouse SMAP2 proteins. Human SMAP-2 Short is SEQ ID NO:4. Human SMAP-2 Long is SEQ ID NO:6. Mouse SMAP-2 is SEQ ID NO:9. Consensus sequence=SEQ ID NO:20.

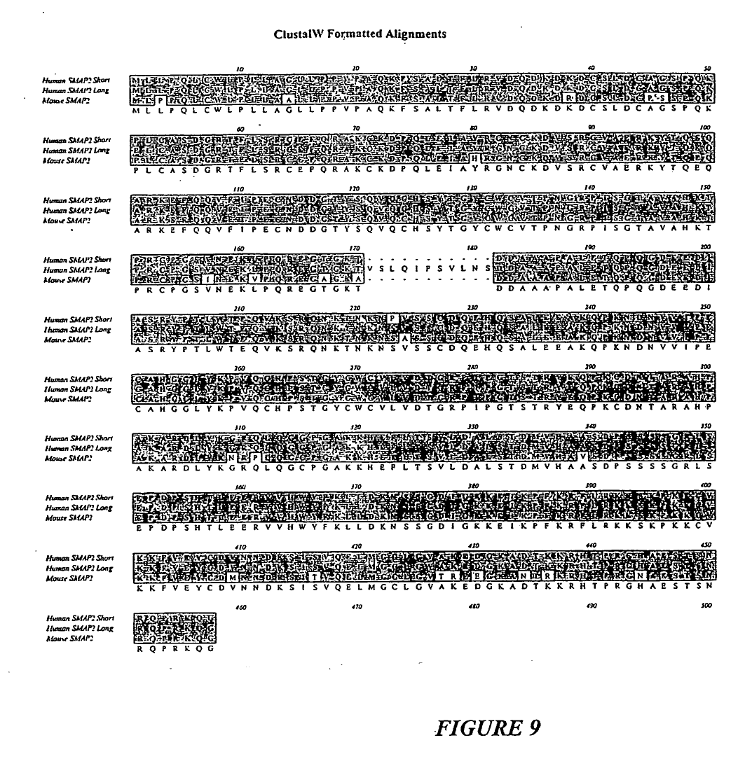

### DETAILED DESCRIPTION

I. Introduction

The present invention demonstrates, surprisingly, that a protease inhibitor, designated SMAP-2, is upregulated in diabetics. SMAP-2 modulates insulin sensitivity in diabetic cells and tissues, including, e.g., adipocytes and skeletal muscle. At least four variants of human SMAP-2 are provided herein (SEQ ID NOs:2, 4, and 6, and the polypeptide encoded by SEQ ID NO:7). In addition, the murine ortholog, designated SMOC2, is provided as SEQ ID NO:9.

Overexpression of human SMAP-2 by either electroporation or adenovirus in 3T3-L1 adipocytes inhibits insulin stimulated GLUT4 translocation and glucose uptake. Thus, overexpression of SMAP-2 in diabetic skeletal muscle decreases the ability of that tissue to dispose of glucose.

SMAP-2 provides a target for inhibition by antagonists. Such antagonists are useful for treating diabetics and pre-diabetic individual. In some embodiments, treatment with SMAP-2 antagonists increases glucose uptake in the skeletal muscle and/or adipocytes. Detection of expression levels of SMAP-2 is useful for diagnosis for diabetic or pre-diabetic individuals.

II. General Recombinant Nucleic Acid Methods for use with the Invention

In numerous embodiments of the present invention, nucleic acids encoding a SMAP-2 of interest will be isolated and cloned using recombinant methods. Such embodiments are used, e.g., to isolate SMAP-2 polynucleotides (e.g., SEQ ID NO:1, SEQ ID NO: 3, SEQ ID NO:5, SEQ ID NO:7 and SEQ ID NO: 8) for protein expression or during the generation of variants, derivatives, expression cassettes, or other sequences derived from a SMAP-2 polypeptide (e.g., SEQ ID NO:2, SEQ ID NO:4, SEQ ID NO:6, SEQ ID NO:9 and the polypeptide encoded by SEQ ID NO:7) to monitor gene expression, for the isolation or detection of SMAP-2 sequences in different species, for diagnostic purposes in a patient, e.g., to detect mutations in SMAP-2 or to-detect expression levels of nucleic acids or polypeptides. In some embodiments, the sequences encoding the polypeptides of the invention are operably linked to a heterologous promoter. In one embodiment, the nucleic acids of the invention are from any mammal, including, e.g., a human, a mouse, a rat, etc.

A. General Recombinant Nucleic Acid Methods

This invention relies on routine techniques in the field of recombinant genetics. Basic texts disclosing the general methods of use in this invention include Sambrook el al., Molecular Cloning, A Laboratory Manual (3rd ed. 2001); Kriegler, Gene Transfer and Expression: A Laboratory Manual (1990); and Current Protocols in Molecular Biology (Ausubel et al., eds., 1994)).

For nucleic acids, sizes are given in either kilobases (kb) or base pairs (bp). These are estimates derived from agarose or acrylamide gel electrophoresis, from sequenced nucleic acids, or from published DNA sequences. For proteins, sizes are given in kilodaltons (kDa) or amino acid residue numbers. Proteins sizes are estimated from gel electrophoresis, from sequenced proteins, from derived amino acid sequences, or from published protein sequences.

Oligonucleotides that are not commercially available can be chemically synthesized according to the solid phase phosphoramidite triester method first described by Beaucage & Caruthers, Tetrahedron Letts. 22:1859-1862 (1981), using an automated synthesizer, as described in Van Devanter et. al., Nucleic Acids Res. 12:6159-6168 (1984). Purification of oligonucleotides is by either native acrylamide gel electrophoresis or by anion-exchange HPLC as described in Pearson & Reanier, J. Chrom. 255:137-149 (1983).

The sequence of the cloned genes and synthetic oligonucleotides can be verified after cloning using, e.g., the chain termination method for sequencing double-stranded templates of Wallace et al., Gene 16:21-26 (1981).

B. Cloning Methods for the Isolation of Nucleotide Sequences Encoding Desired Proteins

In general, the nucleic acids encoding the subject proteins are cloned from DNA sequence libraries that are made to encode cDNA or genomic DNA. The particular sequences can be located by hybridizing with an oligonucleotide probe, the sequence of which can be derived from the sequences disclosed herein. The sequences disclosed herein also provide a reference for PCR primers and defines suitable regions for isolating SMAP-2 specific probes. Alternatively, where the sequence is cloned into an expression library, the expressed recombinant protein can be detected immunologically with antisera or purified antibodies made against a polypeptide of interest, including those disclosed herein.

Methods for making and screening genomic and cDNA libraries are well known to those of skill in the art (see, e.g., Gubler and Hoffman Gene 25:263-269 (1983); Benton and Davis Science, 196:180-182 (1977); and Sambrook, supra). Heart and omental cells are an example of suitable cells to isolate SMAP-2 RNA.

Briefly, to make the cDNA library, one should choose a source that is rich in mRNA. The mRNA can then be made into cDNA, ligated into a recombinant vector, and transfected into a recombinant host for propagation, screening and cloning. For a genomic library, the DNA is extracted from a suitable tissue and either mechanically sheared or enzymatically digested to yield fragments of preferably about 5-100 kb. The fragments are then separated by gradient centrifugation from undesired sizes and are constructed in bacteriophage lambda vectors. These vectors and phage are packaged in vitro, and the recombinant phages are analyzed by plaque hybridization. Colony hybridization is carried out as generally described in Grunstein et al., Proc. Natl. Acad. Sci. USA., 72:3961-3965 (1975).

An alternative method combines the use of synthetic oligonucleotide primers with polymerase extension on an mRNA or DNA template. Suitable primers can be designed from specific SMAP-2 sequences disclosed herein. The polymerase chain reaction (PCR) method amplifies the nucleic acids encoding the protein of interest directly from mRNA, cDNA, genomic libraries or cDNA libraries. Restriction endonuclease sites can be incorporated into the primers. Polymerase chain reaction or other in vitro amplification methods may also be useful, for example, to clone nucleic acids encoding specific proteins and express said proteins, to synthesize nucleic acids that will be used as probes for detecting the presence of mRNA encoding a polypeptide of the invention in physiological samples, for nucleic acid sequencing, or for other purposes (see, U.S. Pat. Nos. 4,683,195 and 4,683,202). Genes amplified by a PCR reaction can be purified from agarose gels and cloned into an appropriate vector.

Appropriate primers and probes for identifying the genes encoding a polypeptide of the invention from mammalian tissues can be derived from the sequences provided herein. For a general overview of PCR, see, Innis et al. PCR Protocols: A Guide to Methods and Applications, Academic Press, San Diego (1990).

Synthetic oligonucleotides can be used to construct genes. This is done using a series of overlapping oligonucleotides, usually 40-120 bp in length, representing both the sense and anti-sense strands of the gene. These DNA fragments are then annealed, ligated and cloned.

A polynucleotide encoding a SMAP-2 polypeptide of the invention can be cloned using intermediate vectors before transformation into mammalian cells for expression. These intermediate vectors are typically prokaryote vectors or shuttle vectors. The proteins can be expressed in either prokaryotes or eukaryotes, using standard methods well known to those of skill in the art.

II. Purification of Proteins of the Invention

Either naturally occurring or recombinant SMAP-2 can be purified for use in functional assays. Naturally occurring SMAP-2 can be purified, e.g., from heart, omental tissues or any other source of a SMAP-2 ortholog. Recombinant polypeptides can be purified from any suitable expression system.

The polypeptides of the invention may be purified to substantial purity by standard techniques, including selective precipitation with such substances as ammonium sulfate; column chromatography, immunopurification methods, and others (see, e.g., Scopes, Protein Purification: Principles and Practice (1982); U.S. Pat. No. 4,673,641; Ausubel et al., supra; and Sambrook et al., supra).

A number of procedures can be employed when recombinant polypeptides arc being purified. For example, proteins having established molecular adhesion properties can be reversibly fused to SMAP-2. With the appropriate ligand, either protein can be selectively adsorbed to a purification column and then freed from the column in a relatively pure form. The fused protein may be then removed by enzymatic activity. Finally polypeptides can be purified using immunoaffinity columns.

A. Purification of Proteins from Recombinant Bacteria

When recombinant proteins are expressed by the transformed bacteria in large amounts, typically after promoter induction, although expression can be constitutive, the proteins may form insoluble aggregates. There are several protocols that are suitable for purification of protein inclusion bodies. For example, purification of aggregate proteins (hereinafter referred to as inclusion bodies) typically involves the extraction, separation and/or purification of inclusion bodies by disruption of bacterial cells typically, but not limited to, by incubation in a buffer of about 100-150 μg/ml lysozyme and 0.1% Nonidet P40, a non-ionic detergent. The cell suspension can be ground using a Polytron grinder (Brinkman Instruments, Westbury, N.Y.). Alternatively, the cells can be sonicated on ice. Alternate methods of lysing bacteria are described in Ausubel et al. and Sambrook et al., both supra, and will be apparent to those of skill in the art.

The cell suspension is generally centrifuged and the pellet containing the inclusion bodies resuspended in buffer which does not dissolve but washes the inclusion bodies, e.g., 20 mM Tris-HCl (pH 7.2), 1 mM EDTA, 150 mM NaCl and 2% Triton-X 100, a non-ionic detergent. It may be necessary to repeat the wash step to remove as much cellular debris as possible. The remaining pellet of inclusion bodies may be resuspended in an appropriate buffer (e.g., 20 mM sodium phosphate, pH 6.8, 150 mM NaCl). Other appropriate buffers will be apparent to those of skill in the art.

Following the washing step, the inclusion bodies are solubilized by the addition of a solvent that is both a strong hydrogen acceptor and a strong hydrogen donor (or a combination of solvents each having one of these properties). The proteins that formed the inclusion bodies may then be renatured by dilution or dialysis with a compatible buffer. Suitable solvents include, but are not limited to, urea (from about 4 M to about 8 M), formamide (at least about 80%, volume/volume basis), and guanidine hydrochloride (from about 4 M to about 8 M). Some solvents that are capable of solubilizing aggregate-forming proteins, such as SDS (sodium dodecyl sulfate) and 70% formic acid, are inappropriate for use in this procedure due to the possibility of irreversible denaturation of the proteins, accompanied by a lack of immunogenicity and/or activity. Although guanidine hydrochloride and similar agents are denaturants, this denaturation is not irreversible and renaturation may occur upon removal (by dialysis, for example) or dilution of the denaturant, allowing re-formation of the immunologically and/or biologically active protein of interest. After solubilization, the protein can be separated from other bacterial proteins by standard separation techniques.

Alternatively, it is possible to purify proteins from bacteria periplasm. Where the protein is exported into the periplasm of the bacteria, the periplasmic fraction of the bacteria can be isolated by cold osmotic shock in addition to other methods known to those of skill in the art (see, Ausubel et al., supra). To isolate recombinant proteins from the periplasm, the bacterial cells are centrifuged to form a pellet. The pellet is resuspended in a buffer containing 20% sucrose. To lyse the cells, the bacteria are centrifuiged and the pellet is resuspended in ice-cold 5 mM MgSO₄ and kept in an ice bath for approximately 10 minutes. The cell suspension is centrifuged and the supernatant decanted and saved. The recombinant proteins present in the supernatant can be separated from the host proteins by standard separation techniques well known to those of skill in the art.

B. Purification of Proteins from Insect Cells

Proteins can also be purified from eukaryotic gene expression systems as described in, e.g., Fernandez and Hoeffler, Gene Expression Systems (1999). In some embodiments, baculovirus expression systems are used to isolate proteins of the invention. Recombinant baculoviruses are generally generated by replacing the polyhedrin coding sequence of a baculovirus with a gene to be expressed (e.g., a SMAP-2 polynucleotide). Viruses lacking the polyhedrin gene have a unique plaque morphology making them easy to recognize. In some embodiments, a recombinant baculovirus is generated by first cloning a polynucleotide of interest into a transfer vector (e.g., a pUC based vector) such that the polynucleotide is operably linked to a polyhedrin promoter. The transfer vector is transfected with wild type DNA into an insect cell (e.g., Sf9, Sf21 or BT1-TN-5B1-4 cells), resulting in homologous recombination and replacement of the polyhedrin gene in the wild type viral DNA with the polynucleotide of interest. Virus can then be generated and plaque purified. Protein expression results upon viral infection of insect cells. Expressed proteins can be harvested from cell supernatant if secreted, or from cell lysates if intracellular. See, e.g., Ausubel et al. and Fernandez and Hoeffler, supra.

C. Standard Protein Separation Techniques for Purifying Proteins

1. Solubility Fractionation

Often as an initial step, and if the protein mixture is complex, an initial salt fractionation can separate many of the unwanted host cell proteins (or proteins derived from the cell culture media) from the recombinant protein of interest. The preferred salt is ammonium sulfate. Ammonium sulfate precipitates proteins by effectively reducing the amount of water in the protein mixture. Proteins then precipitate on the basis of their solubility. The more hydrophobic a protein is, the more likely it is to precipitate at lower ammonium sulfate concentrations. A typical protocol is to add saturated ammonium sulfate to a protein solution so that the resultant ammonium sulfate concentration is between 20-30%. This will precipitate the most hydrophobic proteins. The precipitate is discarded (unless the protein of interest is hydrophobic) and ammonium sulfate is added to the supernatant to a concentration known to precipitate the protein of interest. The precipitate is then solubilized in buffer and the excess salt removed if necessary, through either dialysis or diafiltration. Other methods that rely on solubility of proteins, such as cold ethanol precipitation, are well known to those of skill in the art and can be used to fractionate complex protein mixtures.

2. Size Differential Filtration

Based on a calculated molecular weight, a protein of greater and lesser size can be isolated using ultrafiltration through membranes of different pore sizes (for example, Amicon or Millipore membranes). As a first step, the protein mixture is ultrafiltered through a membrane with a pore size that has a lower molecular weight cut-off than the molecular weight of the protein of interest. The retentate of the ultrafiltration is then ultrafiltered against a membrane with a molecular cut off greater than the molecular weight of the protein of interest. The recombinant protein will pass through the membrane into the filtrate. The filtrate can then be chromatographed as described below.

3. Column Chromatography

The proteins of interest can also be separated from other proteins on the basis of their size, net surface charge, hydrophobicity and affinity for ligands. In addition, antibodies raised against proteins can be conjugated to column matrices and the proteins immunopurified. All of these methods are well known in the art.

Immunoaffinity chromatography using antibodies raised to a variety of affinity tags such as hemagglutinin (HA), FLAG, Xpress, Myc, hexahistidine (His) (SEQ ID NO:13), glutathione S transferase (GST) and the like can be used to purify polypeptides. The His tag will also act as a chelating agent for certain metals (e.g., Ni) and thus the metals can also be used to purify His-containing polypeptides. After purification, the tag is optionally removed by specific proteolytic cleavage.

It will be apparent to one of skill that chromatographic techniques can be performed at any scale and using equipment from many different manufacturers (e.g., Pharmacia Biotech).

IV. Detection of Polynucleotides of the Invention

Those of skill in the art will recognize that detection of expression of SMAP-2 polynucleotides has many uses. For example, as discussed herein, detection of SMAP-2 levels in a patient is useful for diagnosing diabetes or a predisposition for at least some of the pathological effects of diabetes. Moreover, detection of gene expression is useful to identify modulators of SMAP-2 expression.

A variety of methods of specific DNA and RNA measurement that use nucleic acid hybridization techniques are known to those of skill in the art (see, Sambrook, supra). Some methods involve electrophoretic separation (e.g., Southern blot for detecting DNA, And Northern blot for detecting RNA), but measurement of DNA and RNA can also be carried out in the absence of electrophoretic separation (e.g., by dot blot). Southern blot of genomic DNA (e.g., from a human) can be used for screening for restriction fragment length polymorphism (RFLP) to detect the presence of a genetic disorder affecting-a SMAP-2 polypeptide of the invention.

The selection of a nucleic acid hybridization format is not critical. A variety of nucleic acid hybridization formats are known to those skilled in the art. For example, common formats include sandwich assays and competition or displacement assays. Hybridization techniques are generally described in Hames and Higgins Nucleic Acid Hybridization, A Practical Approach, IRL Press (1985); Gall and Pardue, Proc. Natl. Acad. Sci. U.S.A., 63:378-383 (1969); and John et al. Nature, 223:582-587 (1969).

Detection of a hybridization complex may require the binding of a signal-generating complex to a duplex of target and probe polynucleotides or nucleic acids. Typically, such binding occurs through ligand and anti-ligand interactions as between a ligand-conjugated probe and an anti-ligand conjugated with a signal. The binding of the signal generation complex is also readily amenable to accelerations by exposure to ultrasonic energy.

The label may also allow indirect detection of the hybridization complex. For example, where the label is a hapten or antigen, the sample can be detected by using antibodies. In these systems, a signal is generated by attaching fluorescent or enzyme molecules to the antibodies or in some cases, by attachment to a radioactive label (see, e.g., Tijssen, “Practice and Theory of Enzyme Immunoassays;” Laboratory Techniques in Biochemistry and Molecular Biology, Burdon and van Knippenberg Eds., Elsevier (1985), pp. 9-20).

The probes are typically labeled either directly, as with isotopes, chromophores, lumiphores, chromogens, or indirectly, such as with biotin, to which a streptavidin complex may later bind. Thus, the detectable labels used in the assays of the present invention can be primary labels (where the label comprises an element that is detected directly or that produces a directly detectable element) or secondary labels (where the detected label binds to a primary label, e.g., as is common in immunological labeling). Typically, labeled signal nucleic acids are used to detect hybridization. Complementary nucleic acids or signal nucleic acids may be labeled by any one of several methods typically used to detect the presence of hybridized polynucleotides. The most common method of detection is the use of autoradiography with ³H, ¹²⁵I, ³⁵S, ¹⁴C, or ³²P-labeled probes or the like.

Other labels include, e.g., ligands that bind to labeled antibodies, fluorophores, chemiluminescent agents, enzymes, and antibodies-that can serve as specific binding pair members for a labeled ligand. An introduction to labels, labeling procedures and detection of labels is found in Polak and Van Noorden Introduction to Immunocytochemistry, 2nd ed. Springer Verlag, NY (1997); and in Haugland Handbook of Fluorescent Probes and Research Chemicals, a combined handbook and catalogue Published by Molecular Probes, Inc. (1996).

In general, a detector that monitors a particular probe or probe combination is used to detect the detection reagent label. Typical detectors include spectrophotometers, phototubes and photodiodes, microscopes, scintillation counters, cameras, film and the like, as well as combinations thereof. Examples of suitable detectors are widely available from a variety of commercial sources known to persons of skill in the art. Commonly, an optical image of a substrate comprising bound labeling moieties is digitized for subsequent computer analysis.

The amount of, for example, an RNA is measured by quantifying the amount of label fixed to the solid support by binding of the detection reagent. Typically, the presence of a modulator during incubation will increase or decrease the amount of label fixed to the solid support relative to a control incubation that does not comprise the modulator, or as compared to a baseline established for a particular reaction type. Means of detecting and quantifying labels are well known to those of skill in the art.

In some embodiments, the target nucleic acid or the probe is immobilized on a solid support. Solid supports suitable for use in the assays of the invention are known to those of skill in the art. As used herein, a solid support is a matrix of material in a substantially fixed arrangement.

A variety of automated solid-phase assay techniques are also appropriate. For instance, very large scale immobilized polymer arrays (VLSIPS™), i.e. Gene Chips or microarrays, available from Affymetrix, Inc. in Santa Clara, Calif. can be used to detect changes in expression levels of a plurality of genes involved in the same regulatory pathways simultaneously. See, Tijssen, supra., Fodor et al. (1991) Science, 251: 767- 777; Sheldon et al. (1993) Clinical Chemistry 39(4): 718-719, and Kozal et al. (1996) Nature Medicine 2(7): 753-759. Similarly, spotted cDNA arrays (arrays of cDNA sequences bound to nylon, glass or another solid support) can also be used to monitor expression of a plurality of genes.

Typically, the array elements are organized in an ordered fashion so that each element is present at a specified location on the substrate. Because the array elements are at specified locations on the substrate, the hybridization patterns and intensities (which together create a unique expression profile) can be interpreted in terms of expression levels of particular genes and can be correlated with a particular disease or condition or treatment. See, e.g., Schena et al., Science 270: 467-470 (1995)) and (Lockhart et al., Nature Biotech. 14: 1675-1680 (1996)).

Hybridization specificity can be evaluated by comparing the hybridization of specificity-control polynucleotide sequences to specificity-control polynucleotide probes that are added to a sample in a known amount. The specificity-control target polynucleotides may have one or more sequence mismatches compared with the corresponding polynucleotide sequences. In this manner, whether only complementary target polynucleotides are hybridizing to the polynucleotide sequences or whether mismatched hybrid duplexes are forming is determined.

Hybridization reactions can be performed in absolute or differential hybridization formats. In the absolute hybridization format, polynucleotide probes from one sample are hybridized to the sequences in a microarray format and signals detected after hybridization complex formation correlate to polynucleotide probe levels in a sample. In the differential hybridization format, the differential expression of a set of genes in two biological samples is analyzed. For differential hybridization, polynucleotide probes from both biological samples are prepared and labeled with different labeling moieties. A mixture of the two labeled polynucleotide probes is added to a microarray. The microarray is then examined under conditions in which the emissions from the two different labels are individually detectable. Sequences in the microarray that are hybridized to substantially equal numbers of polynucleotide probes derived from both biological samples give a distinct combined fluorescence (Shalon et al. PCT publication WO95/35505). In some embodiments, the labels are fluorescent labels with distinguishable emission spectra, such as Cy3 and Cy5 fluorophores.

After hybridization, the microarray is washed to remove nonhybridized nucleic acids and complex formation between the hybridizable array elements and the polynucleotide probes is detected. Methods for detecting complex formation are well known to those skilled in the art. In some embodiments, the polynucleotide probes are labeled with a fluorescent label and measurement of levels and patterns of fluorescence indicative of complex formation is accomplished by fluorescence microscopy, such as confocal fluorescence microscopy.

In a differential hybridization experiment, polynucleotide probes from two or more different biological samples are labeled with two or more different fluorescent labels with different emission wavelengths. Fluorescent signals are detected separately with different photomultipliers set to detect specific wavelengths. The relative abundances/expression levels of the polynucleotide probes in two or more samples are obtained.

Typically, microarray fluorescence intensities can be normalized to take into account variations in hybridization intensities when more than one microarray is used under similar test conditions. In some embodiments, individual polynucleotide probe/target complex hybridization intensities are normalized using the intensities derived from internal normalization controls contained on each microarray.

Detection of nucleic acids can also be accomplished, for example, by using a labeled detection moiety that binds specifically to duplex nucleic acids (e.g., an antibody that is specific for RNA-DNA duplexes). One example uses an antibody that recognizes DNA-RNA heteroduplexes in which the antibody is linked to an enzyme (typically by recombinant or covalent chemical bonding). The antibody is detected when the enzyme reacts with its substrate, producing a detectable product. Coutlee et al. (1989) Analytical Biochemistry 181:153-162; Bogulavski (1986) et al. J. Immunol. Methods 89:123-130; Prooijen-Knegt (1982) Exp. Cell Res. 141:397-407; Rudkin (1976) Nature 265:472-473, Stollar (1970) PNAS 65:993-1000; Ballard (1982) Mol. Immunol. 19:793-799; Pisetsky and Caster (1982) Mol. Immunol. 19:645-650; Viscidi et al. (1988) J. Clin. Microbial. 41:199-209; and Kiney et al. (1989) J. Clin. Microbiol. 27:6-12 describe antibodies to RNA duplexes, including homo and heteroduplexes. Kits comprising antibodies specific for DNA:RNA hybrids are available, e.g., from Digene Diagnostics, Inc. (Beltsville, Md.).

In addition to available antibodies, one of skill in the art can easily make antibodies specific for nucleic acid duplexes using existing techniques, or modify those antibodies that are commercially or publicly available. In addition to the art referenced above, general methods for producing polyclonal and monoclonal antibodies are known to those of skill in the art (see, e.g., Paul (ed) Fundamental Immunology, Third Edition Raven Press, Ltd., NY (1993); Coligan Current Protocols in Immunology Wiley/Greene, NY (1991); Harlow and Lane Antibodies: A Laboratory Manual Cold Spring Harbor Press, NY (1989); Stites et al (eds.) Basic and Clinical Immunology (4th ed.) Lange Medical Publications, Los Altos, Calif., and references cited therein; Goding Monoclonal Antibodies: Principles and Practice (2d ed.) Academic Press, New York, N.Y., (1986); and Kohler and Milstein Nature 256: 495-497 (1975)). Other suitable techniques for antibody preparation include selection of libraries of recombinant antibodies in phage or similar vectors (see, Huse et al. Science 246:1275-1281(1989); and Ward et al. Nature 341:544-546 (1989)). Specific monoclonal and polyclonal antibodies and antisera will usually bind with a K_{D} of at least about 0.1 μM, preferably at least about 0.01 μM or better, and most typically and preferably, 0.001 μM or better.

The nucleic acids used in this invention can be either positive or negative probes. Positive probes bind to their targets and the presence of duplex formation is evidence of the presence of the target. Negative probes fail to bind to the suspect target and the absence of duplex formation is evidence of the presence of the target. For example, the use of a wild type specific nucleic acid probe or PCR primers may serve as a negative probe in an assay sample where only the nucleotide sequence of interest is present.

The sensitivity of the hybridization assays may be enhanced through use of a nucleic acid amplification system that multiplies the target nucleic acid being detected. Examples of such systems include the polymerase chain reaction (PCR) system and the ligase chain reaction (LCR) system. Other methods recently described in the art are the nucleic acid sequence based amplification (NASBA, Cangene, Mississauga, Ontario) and Q Beta Replicase systems. These systems can be used to directly identify mutants where the PCR or LCR primers are designed to be extended or ligated only when a selected sequence is present. Alternatively, the selected sequences can be generally amplified using, for example, nonspecific PCR primers and the amplified target region later probed for a specific sequence indicative of a mutation. It is understood that various detection probes, including Taqman and molecular beacon probes can be used to monitor amplification reaction products, e.g., in real time.

An alternative means for determining the level of expression of the nucleic acids of the present invention is in situ hybridization. In situ hybridization assays are well known and are generally described in Angerer et al., Methods Enzymol. 152:649-660 (1987). In an in situ hybridization assay, cells, preferentially human cells from the cerebellum or the hippocampus, are fixed to a solid support, typically a glass slide. If DNA is to be probed, the cells are denatured with heat or alkali. The cells are then contacted with a hybridization solution at a moderate temperature to permit annealing of specific probes that are labeled. The probes are preferably labeled with radioisotopes or fluorescent reporters.

Single nucleotide polymorphism (SNP) analysis is also useful for detecting differences between alleles of SMAP-2 genes. SMAP-2 linked SNPs are useful, for instance for diagnosis of SMAP-2-linked diseases (e.g., diabetes or a predisposition for such diseases) in a patient. For example, if an individual carries at least one allele of a SMAP-2-linked SNP, the individual is likely predisposed for one or more of those diseases. If the individual is homozygous for a disease-linked SMAP-2 SNP, the individual is particularly predisposed for SMAP-2-linked disease (e.g., diabetes). In some embodiments, the SNP associated with the SMAP-2-linked disease is located within 300,000; 200,000; 100,000; 75,000; 50,000; 10,000, 5,000; 2,000 or 1,000 base pairs of a polynucleotide encoding SMAP-2.

Various real-time PCR methods including, e.g., Taqman or molecular beacon-based assays (e.g., U.S. Pat. Nos. 5,210,015; 5,487,972; Tyagi et al., Nature Biotechnology 14:303 (1996); and PCT WO 95/13399 are useful to monitor for the presence of absence of a SNP. Additional SNP detection methods include, e.g., DNA sequencing, sequencing by hybridization, dot blotting, oligonucleotide array (DNA Chip) hybridization analysis, or are described in, e.g., U.S. Pat. No. 6,177,249; Landegren et al., Genome Research, 8:769-776 (1998); Botstein et al., Am J Human Genetics 32:314-331 (1980); Meyers et al., Methods in Enzymology 155:501-527 (1987); Keen et al., Trends in Genetics 7:5 (1991); Myers et al., Science 230:1242-1246 (1985); and Kwok et al., Genomics 23:138-144 (1994).

V. Immunological Detection of SMAP-2

In addition to the detection of SMAP-2 genes and gene expression using nucleic acid hybridization technology, one can also use immunoassays to detect SMAP-2 polypeptides. Immunoassays can be used to qualitatively or quantitatively analyze SMAP-2. A general overview of the applicable technology can be found in Harlow & Lane, Antibodies: A Laboratory Manual (1988). Those of skill in the art will recognize that antibodies useful for detection purposes are also useful as therapeutics, i.e., SMAP-2 antagonists.

A. Antibodies to Target Proteins or Other Immunogens

Methods for producing polyclonal and monoclonal antibodies that react specifically with a protein of interest or other immunogen are known to those of skill in the art (see, e.g., Coligan, supra; and Harlow and Lane, supra; Stites et al., supra and references cited therein; Goding, supra; and Kohler and Milstein Nature, 256:495-497 (1975)). Such techniques include antibody preparation by selection of antibodies from libraries of recombinant antibodies in phage or similar vectors (see, Huse et al, supra; and Ward et al., supra). For example, in order to produce antisera for use in an immunoassay, the protein of interest or an antigenic fragment thereof, is isolated as described herein. For example, a recombinant protein is produced in a transformed cell line. An inbred strain of mice or rabbits is immunized with the protein using a standard adjuvant, such as Freund's adjuvant, and a standard immunization protocol. Alternatively, a synthetic peptide derived from the SMAP-2 sequences disclosed herein is conjugated to a carrier protein and used as an immunogen.

Polyclonal sera are collected and titered against the immunogen in an immunoassay, for example, a solid phase immunoassay with the immunogen immobilized on a solid support. Polyclonal antisera with a titer of 10⁴ or greater are selected and tested for their cross-reactivity against non-SMAP-2 proteins or even other homologous proteins from other organisms, using a competitive binding immunoassay. Specific monoclonal and polyclonal antibodies and antisera will usually bind with a K_{D} of at least about 0.1 mM, more usually at least about 1 μM, preferably at least about 0.1 μM or better, and most preferably, 0.01 μM or better.

A number of proteins of the invention comprising immunogens may be used to produce antibodies specifically or selectively reactive with the proteins of interest. Recombinant protein is the preferred immunogen for the production of monoclonal or polyclonal antibodies. Naturally occurring protein may also be used either in pure or impure form. Synthetic peptides made using the protein sequences described herein may also be used as an immunogen for the production of antibodies to the protein. Recombinant protein can be expressed in eukaryotic or prokaryotic cells and purified as generally described supra. The product is then injected into an animal capable of producing antibodies. Either monoclonal or polyclonal antibodies may be generated for subsequent use in immunoassays to measure the protein.

Methods of production of polyclonal antibodies are known to those of skill in the art. In brief, an immunogen, preferably a purified protein, is mixed with an adjuvant and animals are immunized. The animal's immune response to the immunogen preparation is monitored by taking test bleeds and determining the titer of reactivity to SMAP-2. When appropriately high titers of antibody to the immunogen are obtained, blood is collected from the animal and antisera are prepared. Further fractionation of the antisera to enrich for antibodies reactive to the protein can be done if desired (see, Harlow and Lane, supra).

Monoclonal antibodies may be obtained using various techniques familiar to those of skill in the art. Typically, spleen cells from an animal immunized with a desired antigen are immortalized, commonly by fusion with a myeloma cell (see, Kohler and Milstein, Eur. J. Immunol. 6:511-519 (1976)). Alternative methods of immortalization include, e.g., transformation with Epstein Barr Virus, oncogenes, or retroviruses, or other methods well known in the art. Colonies arising from single immortalized cells are screened for production of antibodies of the desired specificity and affinity for the antigen, and yield of the monoclonal antibodies produced by such cells may be enhanced by various techniques, including injection into the peritoneal cavity of a vertebrate host. Alternatively, one may isolate DNA sequences that encode a monoclonal antibody or a binding fragment thereof by screening a DNA library from human B cells according to the general protocol outlined by Huse et al., supra.

Once target immunogen-specific antibodies are available, the immunogen can be measured by a variety of immunoassay methods with qualitative and quantitative results available to the clinician. For a review of immunological and immunoassay procedures in general see, Stites, supra. Moreover, the immunoassays of the present invention can be performed in any of several configurations, which are reviewed extensively in Maggio Enzyme Immunoassay, CRC Press, Boca Raton, Fla. (1980); Tijssen, supra; and Harlow and Lane, supra.

Immunoassays to measure target proteins in a human sample may use a polyclonal antiserum that was raised to the protein (e.g., SMAP-2) or a fragment thereof. This antiserum is selected to have low cross-reactivity against non-SMAP-2 proteins and any such cross-reactivity is removed by immunoabsorption prior to use in the immunoassay.

B. Immunological Binding Assays

In some embodiments, a protein of interest is detected and/or quantified using any of a number of well-known immunological binding assays (see, e.g., U.S. Pat. Nos. 4,366,241; 4,376,110; 4,517,288; and 4,837,168). For a review of the general immunoassays, see also Asai Methods in Cell Biology Volume 37: Antibodies in Cell Biology, Academic Press, Inc. NY (1993); Stites, supra. Immunological binding assays (or immunoassays) typically utilize a “capture agent” to specifically bind to and often immobilize the analyte (SMAP-2, or antigenic subsequences thereof). The capture agent is a moiety that specifically binds to the analyte. In a preferred embodiment, the capture agent is an antibody that specifically binds, for example, a SMAP-2 polypeptide of the invention. The antibody (e.g., anti-SMAP-2 antibody) may be produced by any of a number of means well known to those of skill in the art and as described above.

Immunoassays also often utilize a labeling agent to bind specifically to and label the binding complex formed by the capture agent and the analyte. The labeling agent may itself be one of the moieties comprising the antibody/analyte complex. Alternatively, the labeling agent may be a third moiety, such as another antibody, that specifically binds to the antibody/protein complex.

In an embodiment, the labeling agent is a second antibody bearing a label. Alternatively, the second antibody may lack a label, but it may, in turn, be bound by a labeled third antibody specific to antibodies of the species from which the second antibody is derived. The second antibody can be modified with a detectable moiety, such as biotin, to which a third labeled molecule can specifically bind, such as enzyme-labeled streptavidin.

Other proteins capable of specifically binding immunoglobulin constant regions such as protein A or protein G, can also be used as the label agents. These proteins are normal constituents of the cell walls of streptococcal bacteria. They exhibit a strong non-immunogenic reactivity with immunoglobulin constant regions from a variety of species (see, generally, Kronval, et al. J. Immunol., 111:1401-1406 (1973); and Akerstrom, et al. J. Immunol., 135:2589-2542 (1985)).

Throughout the assays, incubation and/or washing steps may be required after each combination of reagents. Incubation steps can vary from about 5 seconds to several hours, e.g., from about 5 minutes to about 24 hours. The incubation time will depend upon the assay format, analyte, volume of solution, concentrations, and the like. Usually, the assays will be carried out at ambient temperature, although they can be conducted over a range of temperatures, such as 10° C. to 40° C.

1. Non-Competitive Assay Formats

Immunoassays for detecting proteins or analytes of interest from tissue samples may be either competitive or noncompetitive. Noncompetitive immunoassays are assays in which the amount of captured protein or analyte is directly measured. In one preferred “sandwich” assay, for example, the capture agent (e.g., SMAP-2 antibodies) can be bound directly to a solid substrate where it is immobilized. These immobilized antibodies then capture the SMAP-2 present in the test sample The SMAP-2 thus immobilized is then bound by a labeling agent, such as a second anti-SMAP-2 antibody bearing a label. Alternatively, the second antibody may lack a label, but it may, in turn, be bound by a labeled third antibody specific to antibodies of the species from which the second antibody is derived. The second can be modified with a detectable moiety, such as biotin, to which a third labeled molecule can specifically bind, such as enzyme-labeled streptavidin.

2. Competitive Assay Formats

In competitive assays, the amount of protein or analyte present in the sample is measured indirectly by measuring the amount of an added (exogenous) protein or analyte (e.g., the SMAP-2 of interest) displaced (or competed away) from a specific capture agent (e.g., antibodies raised to SMAP-2) by the protein or analyte present in the sample. The amount of immunogen bound to the antibody is inversely proportional to the concentration of immunogen present in the sample. In a particularly preferred embodiment, the antibody is immobilized on a solid substrate. The amount of analyte may be detected by providing a labeled analyte molecule. It is understood that labels can include, e.g., radioactive labels as well as peptide or other tags that can be recognized by detection reagents such as antibodies.

Immunoassays in the competitive binding format can be used for cross-reactivity determinations. For example, the protein encoded by the sequences described herein can be immobilized on a solid support. Proteins are added to the assay and compete with the binding of the antisera to the immobilized antigen. The ability of the above proteins to compete with the binding of the antisera to the immobilized protein is compared to that of the protein encoded by any of the sequences described herein. The percent cross-reactivity for the above proteins is calculated, using standard calculations. Those antisera with less than 10% cross-reactivity with each of the proteins listed above are selected and pooled. The cross-reacting antibodies are optionally removed from the pooled antisera by immunoabsorption with the considered proteins, e.g., distantly related homologs.

The immunoabsorbed and pooled antisera are then used in a competitive binding immunoassay as described above to compare a second protein, thought to be perhaps a protein of the present invention, to the immunogen protein. In order to make this comparison, the two proteins are each assayed at a wide range of concentrations and the amount of each protein required to inhibit 50% of the binding of the antisera to the immobilized protein is determined. If the amount of the second protein required is less than 10 times the amount of the protein partially encoded by a sequence herein that is required, then the second protein is said to specifically bind to an antibody generated to an immunogen consisting of the target protein.

3. Other Assay Formats

In a particularly preferred embodiment, western blot (immunoblot) analysis is used to detect and quantify the presence of a SMAP-2 of the invention in the sample, The technique generally comprises separating sample proteins by gel electrophoresis on the basis of molecular weight, transferring the separated proteins to a suitable solid support (such as, e.g., a nitrocellulose filter, a nylon filter, or a derivatized nylon filter) and incubating the sample with the antibodies that specifically bind the protein of interest. For example, the anti-SMAP-2 antibodies specifically bind to the SMAP-2 on the solid support. These antibodies may be directly labeled or alternatively may be subsequently detected using labeled antibodies (e.g., labeled sheep anti-mouse antibodies) that specifically bind to the antibodies against the protein of interest.

Other assay formats include liposome immunoassays (LIA), which use liposomes designed to bind specific molecules (e.g., antibodies) and release encapsulated reagents or markers. The released chemicals are then detected according to standard techniques (see, Monroe et al. (1986) Amer. Clin. Prod. Rev. 5:34-41).

4. Labels

The particular label or detectable group used in the assay is not a critical aspect of the invention, as long as it does not significantly interfere with the specific binding of the antibody used in the assay. The detectable group can be any material having a detectable physical or chemical property. Such detectable labels have been well-developed in the field of immunoassays and, in general, most labels useful in such methods can be applied to the present invention. Thus, a label is any composition detectable by spectroscopic, photochemical, biochemical, immunochemical, electrical, optical or chemical means. Useful labels in the present invention include magnetic beads (e.g., Dynabeads™), fluorescent dyes (e.g., fluorescein isothiocyanate, Texas red, rhodamine, and the like), radiolabels (e.g., ³H, ¹²⁵I, ³⁵S, ¹⁴C, or ³²P), enzymes (e.g., horse radish peroxidase, alkaline phosphatase and others commonly used in an ELISA), and colorimetric labels such as colloidal gold or colored glass or plastic (e.g., polystyrene, polypropylene, latex, etc.) beads.

The label may be coupled directly or indirectly to the desired component of the assay according to methods well known in the art. As indicated above, a wide variety of labels may be used, with the choice of label depending on the sensitivity required the ease of conjugation with the compound, stability requirements, available instrumentation, and disposal provisions.

Non-radioactive labels are often attached by indirect means. The molecules can also be conjugated directly to signal generating compounds, e.g., by conjugation with an enzyme or fluorescent compound. A variety of enzymes and fluorescent compounds can be used with the methods of the present invention and are well-known to those of skill in the art (for a review of various labeling or signal producing systems which may be used, see, e.g., U.S. Pat. No. 4,391,904).

Means of detecting labels are well known to those of skill in the art. Thus, for example, where the label is a radioactive label, means for detection include a scintillation counter or photographic film as in autoradiography. Where the label is a fluorescent label, it may be detected by exciting the fluorochrome with the appropriate wavelength of light and detecting the resulting fluorescence. The fluorescence may be detected visually, by means of photographic film, by the use of electronic detectors such as charge coupled devices (CCDs) or photomultipliers and the like. Similarly, enzymatic labels may be detected by providing the appropriate substrates for the enzyme and detecting the resulting reaction product. Finally simple colorimetric labels may be detected directly by observing the color associated with the label. Thus, in various dipstick assays, conjugated gold often appears pink, while various conjugated beads appear the color of the bead.

Some assay formats do not require the use of labeled components. For instance, agglutination assays can be used to detect the presence of the target antibodies. In this case, antigen-coated particles are agglutinated by samples comprising the target antibodies. In this format, none of the components need to be labeled and the presence of the target antibody is detected by simple visual inspection.

VI. Identification of Modulators of SMAP-2

Modulators of SMAP-2, i.e. agonists or antagonists of SMAP-2 activity, or SMAP-2 polypeptide or polynucleotide expression, are useful for treating a number of human diseases, including diabetes. For example, administration of SMAP-2 antagonists can be used to treat diabetic patients or individuals with insulin resistance to prevent progression, and therefore symptoms, associated with diabetes.

A. Agents that Modulate SMAP-2

The agents tested as modulators of SMAP-2 can be any small chemical compound, or a biological entity, such as a protein, sugar, nucleic acid or lipid. Typically, test compounds will be small chemical molecules and peptides. Essentially any chemical compound can be used as a potential modulator or ligand in the assays of the invention, although most often compounds that can be dissolved in aqueous or organic (especially DMSO-based) solutions are used. The assays are designed to screen large chemical libraries by automating the assay steps and providing compounds from any convenient source to assays, which are typically run in parallel (e.g., in microtiter formats on microtiter plates in robotic assays). Modulators also include agents designed to reduce the level of SMAP-2 mRNA (e.g. antisense molecules, ribozymes, DNAzymes, small inhibitory RNAs and the like) or the level of translation from an mRNA (e.g., translation blockers such as antisense molecules that are complementary to translation start or other sequences on an mRNA molecule). It will be appreciated that there are many suppliers of chemical compounds, including Sigma (St. Louis, Mo.), Aldrich (St. Louis, Mo.), Sigma-Aldrich (St. Louis, Mo.), Fluka Chemika-Biochemica Analytika (Buchs, Switzerland) and the like.

In some embodiments, high throughput screening methods involve providing a combinatorial chemical or peptide library containing a large number of potential therapeutic compounds (potential modulator compounds). Such “combinatorial chemical libraries” or “ligand libraries” are then screened in one or more assays, as described herein, to identify those library members (particular chemical species or subclasses) that display a desired characteristic activity. The compounds thus identified can serve as conventional “lead compounds” or can themselves be used as potential or actual therapeutics.

A combinatorial chemical library is a collection of diverse chemical compounds generated by either chemical synthesis or biological synthesis, by combining a number of chemical “building blocks” such as reagents. For example, a linear combinatorial chemical library such as a polypeptide library is formed by combining a set of chemical building blocks (amino acids) in every possible way for a given compound length (i.e., the number of amino acids in a polypeptide compound). Millions of chemical compounds can be synthesized through such combinatorial mixing of chemical building blocks.

Preparation and screening of combinatorial chemical libraries is well known to those of skill in the art. Such combinatorial chemical libraries include, but are not limited to, peptide libraries (see, e.g., U.S. Pat. No. 5,010,175, Furka, Int. J. Pept. Prot. Res. 37:487493 (1991) and Houghton et al., Nature 354:84-88 (1991)). Other chemistries for generating chemical diversity libraries can also be used. Such chemistries include, but are not limited to: peptoids (e.g., PCT Publication No. WO 91/19735), encoded peptides (e.g., PCT Publication WO 93/20242), random bio-oligomers (e.g., PCT Publication No. WO 92/00091), benzodiazepines (e.g., U.S. Pat. No. 5,288,514) diversomers such as hydantoins, benzodiazepines and dipeptides (Hobbs et al., Proc. Nat. Acad. Sci. USA 90:6909-6913 (1993), vinylogous polypeptides (Hagihara et al., J. Amer. Chem. Soc. 114:6568 (1992)), nonpeptidal peptidomimetics with glucose scaffolding (Hirschmann et al., J. Amer. Chem. Soc. 114:9217-9218 (1992)), analogous organic syntheses of small compound libraries (Chen et al., J. Amer. Chem. Soc. 116:2661 (1994)), oligocarbamates (Cho et al., Science 261:1303 (1993)), and/or peptidyl phosphonates (Campbell et al., J. Org. Chem. 59:658 (1994)), nucleic acid libraries (see Ausubel, Berger and Sambrook, all supra), peptide nucleic acid libraries (see, e.g., U.S. Pat. No. 5,539,083), antibody libraries (see, e.g., Vaughn et al., Nature Biotechnology, 14(3):309-314 (1996) and PCT/US96/10287), carbohydrate libraries (see, e.g., Liang et al., Science, 274:1520-1522 (1996) and U.S. Pat. No. 5,593,853), small organic molecule libraries (see, e.g., benzodiazepines, Baum C&EN, January 18, page 33 (1993); isoprenoids, U.S. Pat. No. 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. No. 5,506,337; benzodiazepines, U.S. Pat. No. 5,288,514, and the like).

Devices for the preparation of combinatorial libraries are commercially available (see, e.g., 357 MPS, 390 MPS, Advanced Chem Tech, Louisville Ky., Symphony, Rainin, Woburn, Mass., 433A Applied Biosystems, Foster City, Calif., 9050 Plus, Millipore, Bedford, Mass.). In addition, numerous combinatorial libraries are themselves commercially available (see, e.g., ComGenex, Princeton, N.J., Tripos, Inc., St. Louis, Mo., 3D Pharmaceuticals, Exton, Pa., Martek Biosciences, Columbia, Md., etc.).

As described above, the present invention also provides antisense molecules for detecting, diagnosing and treating SMAP-2-related conditions. Antisense compounds include ribozymes, external guide sequence (EGS) oligonucleotides (oligozymes), and other short catalytic RNAs or catalytic oligonucleotides that hybridize to the target nucleic acid and modulate its expression. Antisense molecules are substantially complementary to a contiguous sequence of a SMAP-2 nucleic acid (e.g., SEQ ID NO:1, SEQ ID NO:3, SEQ ID NO:5, SEQ ID NO:7 or SEQ ID NO:8). Antisense polynucleotides of the invention are characterized by their ability to specifically hybridize to naturally-occurring and synthetic SMAP-2 nucleic acids, including any upstream, flanking, noncoding, and transcriptional control elements, pre-mRNA, mRNA, cDNA and the like. The antisense polynucleotides of the invention are typically at least 7, 8, 9, 10, 11, 12, 13, 14, 15, or 18 nucleotides in length, at least 20, 30, 40, 50 70, 100 nucleotides in length, or the full length of a SMAP-2 mRNA.

In certain embodiments, the antisense sequence is exactly complementary to the target sequence. The antisense polynucleotides may also include, however, nucleotide substitutions, additions, deletions, transitions, transpositions, or modifications, or other nucleic acid sequences or non-nucleic acid moieties so long as specific binding to the relevant target sequence corresponding to SMAP-2 RNA or its gene is retained as a functional property of the polynucleotide.

Antisense polynucleotides comprising backbone analogues such as phosphodiester, phosphorothioate, phosphorodithioate, methylphosphonate, phosphoramidate, alkyl phosphotriester, sulfamate, 3′-thioacetal, methylene(methylimino), 3′-N-carbamate, morpholino carbamate, chiral-methyl phosphonates, nucleotides with short chain alkyl or cycloalkyl intersugar linkages, short chain heteroatomic or heterocyclic intersugar (“backbone”) linkages, or CH2-NH—O—CH2, CH2-N(CH3)-OCH2, CH2-O—N(CH3)-CH2, CH2-N(CH3)-N(CH3)-CH2 and O—N(CH3)-CH2-CH2 backbones (where phosphodiester is O—P—O—CH2), or mixtures of the same are provided. Also useful are oligonucleotides having morpholino backbone structures (U.S. Pat. No. 5,034,506).

Without intending to be limited to any particular mechanism, it is believed that antisense polynucleotides bind to, and interfere with the translation of, the sense SMAP-2 mRNA. Alternatively, the antisense molecule may render the SMAP-2 mRNA susceptible to nuclease digestion, interfere with transcription, interfere with processing, localization or otherwise with RNA precursors (“pre-mRNA”), repress transcription of mRNA from the SMAP-2 gene, or act through some other mechanism. However, the particular mechanism by which the antisense molecule reduces SMAP-2 expression is not critical.

B. Methods of Screening for Modulators of SMAP-2

A number of different screening protocols can be used to identify agents that modulate the level of expression or activity of SMAP-2 in cells, including mammalian cells such as human cells. In general terms, the screening methods involve screening a plurality of agents to identify an agent that modulates the activity of SMAP-2 by, e.g., binding to a SMAP-2 polypeptide, preventing SMAP-2 from binding a protease, increasing association of an inhibitor or activator with SMAP-2, or activating or inhibiting expression of SMAP-2.

1. SMAP-2 Binding Assays

Preliminary screens can be conducted by screening for agents capable of binding to SMAP-2, as at least some of the agents so identified are likely SMAP-2 modulators. Binding assays are also useful, e.g., for identifying endogenous proteins that interact with SMAP-2. For example, antibodies, receptors., proteases or other molecules that bind SMAP-2 can be identified in binding assays.

Binding assays usually involve contacting a SMAP-2 protein with one or more test agents and allowing sufficient time for the protein and test agents to form a binding complex. Any binding complexes formed can be detected using any of a number of established analytical techniques. Protein binding assays include, but are not limited to, methods that measure co-precipitation or co-migration on non-denaturing SDS-polyacrylamide gels, and co-migration on Western blots (see, e.g., Bennet, J. P. and Yamamura, H. I. (1985) “Neurotransmitter, Hormone or Drug Receptor Binding Methods,” in Neurotransmitter Receptor Binding (Yamamura, H. I., et al., eds.), pp. 61-89. Other binding assays involve the use of mass spectrometry or NMR techniques to identify molecules bound to SMAP-2 or displacement of labeled substrates. The SMAP-2 proteins utilized in such assays can be naturally expressed, cloned or synthesized.

In addition, mammalian or yeast two-hybrid approaches (see, e.g., Bartel, P. L. et. al. Methods Enzymol, 254:241 (1995)) can be used to identify polypeptides or other molecules that interact or bind when expressed together in a host cell.

2. SMAP-2 Activity

SMAP-2 and its alleles and polymorphic variants are protease inhibitors. The activity of SMAP-2 polypeptides can be assessed using a variety of in vitro and in vivo assays to determine functional, chemical, and physical effects, e.g., measuring the ability of SMAP-2 to inhibit protease activity or by measuring the effect of SMAP-2 modulation on insulin sensitivity. For example, a candidate SMAP-2 modulator can be added to a mixture comprising a SMAP2-expressing cell capable of uptaking glucose in response to insulin. Such assays can be used to test for inhibitors and activators of SMAP-2. Such modulators of transduction activity are useful for controlling cell signaling and treating diabetes.

The SMAP-2 of the assay can be selected from a polypeptide having a sequence or subsequence of SEQ ID NOs:2, 4, 6, 9, the polypeptide encoded by SEQ ID NO:7, or conservatively modified variants thereof. Alternatively, the SMAP-2 of the assay will be derived from a eukaryote. Generally, orthologous SMAP-2 sequences are substantially identical to SEQ ID NOs: 2, 4, 6, or 9. Optionally, the polypeptide of the assays will comprise a fragment of SMAP-2 comprising one or more domains of SMAP-2, such as an export signal sequence, one or two EF calcium binding hands, a Kazal domain and one or two thyroglobulin-1 repeat domains and the like. Either SMAP-2 or a domain thereof can be covalently linked to a heterologous protein to create a chimeric protein used in the assays described herein.

Modulators of SMAP-2 activity are tested using SMAP-2 polypeptides as described above, either recombinant or naturally occurring. The protein can be isolated, expressed in a cell, expressed in tissue or in an animal, either recombinant or naturally occurring. For example, tissue slices, dissociated cells, e.g., from tissues expressing SMAP-2, transformed cells, or membranes can be used. Modulation is tested using one of the in vitro or in vivo assays described herein. Furthermore, protease-binding domains of the protein of interest can be used in vitro in soluble or solid state reactions to assay for modulator binding.

Insulin sensitivity of the cell can be determined in any number of ways. For example, GLUT4 translocation can be used to monitor the effects of a candidate SMAP-2 modulator on cells. GLUT4 translocation can be determined by monitoring the quantity of GLUT4 associated with the plasma membrane. For example, following a stimulus, plasma membrane fractions can be isolated and the quantity of GLUT4 in the fraction can be measured. See, e.g., Song et al., J. Biol. Chem. 276:34651 (2001). Alternatively, GLUT4-myc fusions can be used to monitor the location of GLUT4.

Glucose transport assays can also be used to measure the effect of an agent on SMAP-2 activity. For example, an agent that interacts with SMAP-2 and increases glucose uptake can be selected for further study.

Samples or assays that are treated with a potential SMAP-2 inhibitor or activator are compared to control samples without the test compound, to examine the extent of modulation. Control samples (untreated with activators or inhibitors) are assigned a relative SMAP-2 activity value of 100. Inhibition of SMAP-2 is achieved when the SMAP-2 activity value relative to the control is about 90%, optionally 50%, optionally 25-0%. Activation of SMAP-2 is achieved when the SMAP-2 activity value relative to the control is 110%, optionally 150%, 200%, 300%, 400%, 500%, or 1000-2000%.

3. Expression Assays

Screening for a compound that modulates the expression of SMAP-2 is also provided. Screening methods generally involve conducting cell-based assays in which test compounds are contacted with one or more cells expressing SMAP-2, and then detecting an increase or decrease in SMAP-2 expression (either transcript or translation product). Assays can be performed with any cells that express SMAP-2.

SMAP-2 expression can be detected in a number of different ways. As described infra, the expression level of SMAP-2 in a cell can be determined by probing the mRNA expressed in a cell with a probe that specifically hybridizes with a transcript (or complementary nucleic acid derived therefrom) of SMAP-2. Probing can be conducted by lysing the cells and conducting Northern blots or without lysing the cells using in situ-hybridization techniques. Alternatively, SMAP-2 protein can be detected using immunological methods in which a cell lysate is probed with antibodies that specifically bind to SMAP-2.

Other cell-based assays involve reporter assays conducted with cells using standard reporter gene assays. These assays can be performed in either cells that do, or do not, express SMAP-2. Some of these assays are conducted with a heterologous nucleic acid construct that includes a SMAP-2 promoter (e.g., comprising SEQ ID NO:10 or SEQ ID NO:11) that is operably linked to a reporter gene that encodes a detectable product. A number of different reporter genes can be utilized. Some reporters are inherently detectable. Examples of such a reporter include luciferase and green fluorescent protein, each of which emits fluorescence that can be detected with a fluorescence detector. Other reporters generate a detectable product. Often such reporters are enzymes. Exemplary enzyme reporters include, but are not limited to, β-glucuronidase; CAT (chloramphenicol acetyl transferase; Alton arid Vapnek (1979) Nature 282:864-869), luciferase, β-galactosidase and alkaline phosphatase (Toh, et al. (1980) Eur. J. Biochem. 182:231-238; and Hall et al. (1983) J. Mol. Appl. Gen. 2:101).

In these assays, cells harboring the reporter construct are contacted with a test compound. Modulated promoter expression is monitored by detecting the level of a detectable reporter. A number of different kinds of SMAP-2 modulators can be identified in this assay. For example, a test compound that inhibits the promoter by binding to it, inhibits the promoter by binding to transcription factors or other regulatory factors, binds to their promoter or triggers a cascade that produces a molecule that inhibits the promoter can be identified. Similarly a test compound that, e.g., activates the promoter by binding to it, activates the promoter by binding to transcription factors or other regulatory factors, binds to their promoter or triggers a cascade that produces a molecule that activates the promoter can also be identified.

The level of expression or activity can be compared to a baseline value. The baseline value can be a value for a control sample or a statistical value that is representative of SMAP-2 expression levels for a control population (e.g., lean individuals as described herein) or cells (e.g., tissue culture cells not exposed to an SMAP-2 modulator). Expression levels can also be determined for cells that do not express SMAP-2 as a negative control. Such cells generally are otherwise substantially genetically the same as the test cells.

A variety of different types of cells can be utilized in the reporter assays. Cells that express an endogenous SMAP-2 include, e.g., heart cells, adipocytes, and skeletal muscle (e.g., L6) cells. Cells that do not endogenously express SMAP-2 can be prokaryotic or eukaryotic. Eukaryotic cells can be any of the cells typically utilized in generating cells that harbor recombinant nucleic acid constructs. Exemplary eukaryotic cells include, but are not limited to, yeast, and various higher eukaryotic cells such as the HEK293, HepG2, COS, CHO and HeLa cell lines.

Various controls can be conducted to ensure that an observed activity is authentic including running parallel reactions with cells that lack the reporter construct or by not contacting a cell harboring the reporter construct with test compound. Compounds can also be further validated as described below.

4. Validation

Agents that are initially identified by any of the foregoing screening methods can be further tested to validate the apparent activity. Preferably such studies are conducted with suitable animal models. The basic format of such methods involves administering a lead compound identified during an initial screen to an animal that serves as a model for humans and then determining if SMAP-2 is in fact modulated. The effect of the compound can be assessed in normal animals, diabetic animals or in diet-induced insulin resistant animals. The blood glucose and insulin levels can be determined in response to administration of candidate SMAP-2 modulators. The animal models utilized in validation studies generally are mammals of any kind. Specific examples of suitable animals include, but are not limited to, primates, mice and rats. For example, monogenic models of diabetes (e.g., ob/ob and db/db mice, Zucker rats and Zucker Diabetic Fatty rats etc) or polygenic models of diabetes (e.g., OLETF rats, GK rats, NSY mice, and KK mice) can be useful for validating SMAP-2 modulation in a diabetic or insulin resistant animals. In addition, transgenic animals expressing human SMAP-2 can be used to further validate drug candidates.

C. Solid Phase and Soluble High Throughput Assays

In the high throughput assays of the invention, it is possible to screen up to several thousand different modulators or ligands in a single day. In particular, each well of a microtiter plate can be used to run a separate assay against a selected potential modulator, or, if concentration or incubation time effects are to be observed, every 5-10 wells can test a single modulator. Thus, a single standard microtiter plate can assay about 100 (e.g., 96) modulators. If 1536 well plates are used, then a single plate can easily assay from about 100 to about 1500 different compounds. It is possible to assay several different plates per day; assay screens for up to about 6,000-20,000 or more different compounds are possible using the integrated systems of the invention. In addition, microfluidic approaches to reagent manipulation can be used.

The molecule of interest (e.g., SMAP-2) can be bound to the solid state component, directly or indirectly, via covalent or non-covalent linkage, e.g., via a tag. The tag can be any of a variety of components. In general, a molecule that binds the tag (a tag binder) is fixed to a solid support, and the tagged molecule of interest (e.g., SMAP-2) is attached to the solid support by interaction of the tag and the tag binder.

A number of tags and tag binders can be used, based upon known molecular interactions well described in the literature. For example, where a tag has a natural binder, for example, biotin, protein A, or protein G, it can be used in conjunction with appropriate tag binders (avidin, streptavidin, neutravidin, the Fc region of an immunoglobulin, poly-His, etc.). Antibodies to molecules with natural binders such as biotin are also widely available and appropriate tag binders (see, SIGMA Immunochemicals 1998 catalogue SIGMA, St. Louis Mo.).

Similarly, any haptenic or antigenic compound can be used in combination with an appropriate antibody to form a tag/tag binder pair. Thousands of specific antibodies are commercially available and many additional antibodies are described in the literature. For example, in one common configuration, the tag is a first antibody and the tag binder is a second antibody that recognizes the first antibody. In addition to antibody-antigen interactions, receptor-ligand interactions are also appropriate as tag and tag-binder pairs, such as agonists and antagonists of cell membrane receptors (e.g., cell receptor-ligand interactions such as transferrin, c-kit, viral receptor ligands, cytokine receptors, chemokine receptors, interleukin receptors, immunoglobulin receptors and antibodies, the cadherin family, the integrin family, the selectin family, and the like; see, e.g., Pigott & Power, The Adhesion Molecule Facts Book I (1993)). Similarly, toxins and venoms, viral epitopes, hormones (e.g., opiates, steroids, etc.), intracellular receptors (e.g., which mediate the effects of various small ligands, including steroids, thyroid hormone, retinoids and vitamin D; peptides), drugs, lectins, sugars, nucleic acids (both linear and cyclic polymer configurations), oligosaccharides, proteins, phospholipids and antibodies can all interact with various cell receptors.

Synthetic polymers, such as polyurethanes, polyesters, polycarbonates, polyureas, polyamides, polyethyleneimines, polyarylene sulfides, polysiloxanes, polyimides, and polyacetates can also form an appropriate tag or tag binder. Many other tag/tag binder pairs are also useful in assay systems described herein, as would be apparent to one of skill upon review of this disclosure.

Common linkers such as peptides, polyethers, and the like can also serve as tags, and include polypeptide sequences, such as poly-Gly sequences of between about 5 and 200 amino acids (SEQ ID NO:14). Such flexible linkers are known to those of skill in the art. For example, poly(ethylene glycol) linkers are available from Shearwater Polymers, Inc., Huntsville, Ala. These linkers optionally have amide linkages, sulfhydryl linkages, or heterofunctional linkages.

Tag binders are fixed to solid substrates using any of a variety of methods currently available. Solid substrates are commonly derivatized or functionalized by exposing all or a portion of the substrate to a chemical reagent that fixes a chemical group to the surface that is reactive with a portion of the tag binder. For example, groups that are suitable for attachment to a longer chain portion would include amines, hydroxyl, thiol, and carboxyl groups. Aminoalkylsilanes and hydroxyalkylsilanes can be used to functionalize a variety of surfaces, such as glass surfaces. The construction of such solid phase biopolymer arrays is well described in the literature (see, e.g., Merrifield, J. Am. Chem. Soc. 85:2149-2154 (1963) (describing solid phase synthesis of, e.g., peptides); Geysen et al, J. Immun. Meth. 102:259-274 (1987) (describing synthesis of solid phase components on pins); Frank and Doring, Tetrahedron 44:60316040 (1988) (describing synthesis of various peptide sequences on cellulose disks); Fodor et al., Science, 251:767-777 (1991); Sheldon et al., Clinical Chemistry 39(4):718-719 (1993); and Kozal et al., Nature Medicine 2(7):753759 (1996) (all describing arrays of biopolymers fixed to solid substrates). Non-chemical approaches for fixing tag binders to substrates include other common methods, such as heat, cross-linking by UV radiation, and the like.

The invention provides in vitro assays for identifying, in a high throughout format, compounds that can modulate the expression or activity of SMAP-2. Control reactions that measure SMAP-2 activity of the cell in a reaction that does not include a potential modulator are optional, as the assays are highly uniform. Such optional control reactions can increase the reliability of the assay. For each of the assay formats described, “no modulator” control reactions that do not include a modulator provide a background level of binding activity.

In some assays it will be desirable to have positive controls. At least two types of positive controls are appropriate. First, a known activator of SMAP-2 of the invention can be incubated with one sample of the assay, and the resulting increase in signal resulting from an increased expression level or activity of SMAP-2 are determined according to the methods herein. Second, a known inhibitor of SMAP-2 can be added, and the resulting decrease in signal for the expression or activity of SMAP-2 can be similarly detected. It will be appreciated that modulators can also be combined with activators or inhibitors to find modulators that inhibit the increase or decrease that is otherwise caused by the presence of the known modulator of SMAP-2.

VII. Compositions, Kits and Integrated Systems

The invention provides compositions, kits and integrated systems for practicing the assays described herein using nucleic-acids encoding the SMAP-2 polypeptides of the invention, or SMAP-2 proteins, anti-SMAP-2 antibodies, etc.

The invention provides assay compositions for use in solid phase assays; such compositions can include, for example, one or more nucleic acids encoding a SMAP-2 immobilized on a solid support, and a labeling reagent. In each case, the assay compositions can also include additional reagents that are desirable for hybridization. Modulators of expression or activity of a SMAP-2 of the invention can also be included in the assay compositions.

The invention also provides kits for carrying out the assays of the invention. The kits typically include a probe that comprises an antibody that specifically binds to SMAP-2 or a polynucleotide sequence encoding a SMAP-2 polypeptide, and a label for detecting the presence of the probe. The kits may include at least one polynucleotide sequence encoding a SMAP-2 polypeptides of the invention. Kits can include any of the compositions noted above, and optionally further include additional components such as instructions to practice a high-throughput method of assaying for an effect on expression of the genes encoding the SMAP-2 polypeptide of the invention, or on activity of the SMAP-2 polypeptides of the invention, one or more containers or compartments (e.g., to hold the probe, labels, or the like), a control modulator of the expression or activity of SMAP-2 polypeptides, a robotic armature for mixing kit components or the like.

The invention also provides integrated systems for high-throughput screening of potential modulators for an effect on the expression or activity of the SMAP-2 polypeptides of the invention. The systems can include a robotic armature which transfers fluid from a source to a destination, a controller which controls the robotic armature, a label detector, a data storage unit which records label detection, and an assay component such as a microtiter dish comprising a well having a reaction mixture or a substrate comprising a fixed nucleic acid or immobilization moiety.

A number of robotic fluid transfer systems are available, or can easily be made from existing components. For example, a Zymate XP (Zymark Corporation; Hopkinton, Mass.) automated robot using a Microlab 2200 (Hamilton; Reno, Nev.) pipetting station can be used to transfer parallel samples to 96 well microtiter plates to set up several parallel simultaneous binding assays.

Optical images viewed (and, optionally, recorded) by a camera or other recording device (e.g., a photodiode and data storage device) are optionally further processed in any of the embodiments herein, e.g., by digitizing the image and storing and analyzing the image on a computer. A variety of commercially available peripheral equipment and software is available for digitizing, storing and analyzing a digitized video or digitized optical image, e.g., using PC (Intel x86 or Pentium chip-compatible DOS®, OS2® WINDOWS®, WINDOWS NT®, WINDOWS95®, WINDOWS98®, or WINDOWS2000® based computers), MACINTOSH®, or UNIX® based (e.g., SUN® work station) computers.

One conventional system carries light from the specimen field to a cooled charge-coupled device (CCD) camera, in common use in the art. A CCD camera includes an array of picture elements (pixels). The light from the specimen is imaged on the CCD. Particular pixels corresponding to regions of the specimen (e.g., individual hybridization sites on an array of biological polymers) are sampled to obtain light intensity readings for each position. Multiple pixels are processed in parallel to increase speed. The apparatus and methods of the invention are easily used for viewing any sample, e.g., by fluorescent or dark field microscopic techniques.

VIII. Administration and Pharmaceutical Compositions

Modulators of SMAP-2 (e.g., antagonists, including SMAP-2 specific antibodies, or agonists) can be administered directly to the mammalian subject for modulation of SMAP-2 activity in vivo. Administration is by any of the routes normally used for introducing a modulator compound into ultimate contact with the tissue to be treated and is well known to those of skill in the art. Although more than one route can be used to administer a particular composition, a particular route can often provide a more immediate and more effective reaction than another route.

The pharmaceutical compositions of the invention may comprise a pharmaceutically acceptable carrier. Pharmaceutically acceptable carriers are determined in part by the particular composition being administered, as well as by the particular method used to administer the composition. Accordingly, there is a wide variety of suitable formulations of pharmaceutical compositions of the present invention (see, e.g., Remington's Pharmaceutical Sciences, 17^{th} ed. 1985)).

The modulators (e.g., agonists or antagonists) of the expression or activity of the SMAP-2, alone or in combination with other suitable components, can be prepared for injection or for use in a pump device. Pump devices (also known as “insulin pumps”) are commonly used to administer insulin to patients and therefore can be easily adapted to include compositions of the present invention. Manufacturers of insulin pumps include Animas, Disetronic and MiniMed.

The modulators (e.g., agonists or antagonists) of the expression or activity of the SMAP-2, alone or in combination with other suitable components, can be made into aerosol formulations (i.e., they can be “nebulized”) to be administered via inhalation. Aerosol formulations can be placed into pressurized acceptable propellants, such as dichlorodifluoromethane, propane, nitrogen, and the like.

Formulations suitable for administration include aqueous and non-aqueous solutions, isotonic sterile solutions, which can contain antioxidants, buffers, bacteriostats, and solutes that render the formulation isotonic, and aqueous and non-aqueous sterile suspensions that can include suspending agents, solubilizers, thickening agents, stabilizers, and preservatives. In the practice of this invention, compositions can be administered, for example, orally, nasally, topically, intravenously, intraperitoneally, or intrathecally. The formulations of compounds can be presented in unit-dose or multi-dose sealed containers, such as ampoules and vials. Solutions and suspensions can be prepared from sterile powders, granules, and tablets of the kind previously described. The modulators can also be administered as part of a prepared food or drug.

The dose administered to a patient, in the context of the present invention should be sufficient to induce a beneficial response in the subject over time. The optimal dose level for any patient will depend on a variety of factors including the efficacy of the specific modulator employed, the age, body weight, physical activity, and diet of the patient, on a possible combination with other drugs, and on the severity of the case of diabetes. It is recommended that the daily dosage of the modulator be determined for each individual patient by those skilled in the art in a similar way as for known insulin compositions. The size of the dose also will be determined by the existence, nature, and extent of any adverse side-effects that accompany the administration of a particular compound or vector in a particular subject.

In determining the effective amount of the modulator to be administered a physician may evaluate circulating plasma levels of the modulator, modulator toxicity, and the production of anti-modulator antibodies. In general, the dose equivalent of a modulator is from about 1 ng/kg to 10 mg/kg for a typical subject.

For administration, SMAP-2 modulators of the present invention can be administered at a rate determined by the LD-50 of the modulator, and the side-effects of the modulator at various concentrations, as applied to the mass and overall health of the subject. Administration can be accomplished via single or divided doses.

The compounds of the present invention (e.g., SMAP-2 specific antibodies) can also be used effectively in combination with one or more additional active agents depending on the desired target therapy (see, e.g., Turner, N. et al. Prog. Drug Res. (1998) 51: 33-94; Haffner, S. Diabetes Care (1998) 21: 160-178; and DeFronzo, R. et al. (eds.), Diabetes Reviews (1997) Vol. 5 No. 4). A number of studies have investigated the benefits of combination therapies with oral agents (see, e.g., Mahler, R., J. Clin. Endocrinol. Melab. (1999) 84: 1165-71; United Kingdom Prospective Diabetes Study Group: UKPDS 28, Diabetes Care (1998) 21: 87-92; Bardin, C. W. (ed.), Current Therapy In Endocrinology And Metabolism, 6th Edition (Mosby—Year Book, Inc., St. Louis, Mo. 1997); Chiasson, J. et al., Ann. Intern. Med. (1994) 121: 928-935; Coniff, R. et al., Clin. Ther. (1997) 19: 16-26; Coniff, R. et al., Am. J. Med. (1995) 98: 443-451; and Iwamoto, Y. et al., Diabet. Med. (1996) 13 365-370; Kwiterovich, P. Am. J. Cardiol (1998) 82(12A): 3U-17U). These studies indicate that modulation of diabetes, among other diseases, can be further improved by the addition of a second agent to the therapeutic regimen. Combination therapy includes administration of a single pharmaceutical dosage formulation that contains a SMAP-2 modulator of the invention and one or more additional active agents, as well as administration of a SMAP-2 modulator and each active agent in its own separate pharmaceutical dosage formulation. For example, a SMAP-2 modulator and a thiazolidinedione can be administered to the human subject together in a single oral dosage composition, such as a tablet or capsule, or each agent can be administered in separate oral dosage formulations. Where separate dosage formulations are used, an SMAP-2 modulator and one or more additional active agents can be administered at essentially the same time (i.e., concurrently), or at separately staggered times (i.e., sequentially). Combination therapy is understood to include all these regimens.

One example of combination therapy can be seen in treating pre-diabetic individuals (e.g., to prevent progression into type 2 diabetes) or diabetic individuals (or treating diabetes and its related symptoms, complications, and disorders), wherein the SMAP-2 modulators can be effectively used in combination with, for example, sulfonylureas (such as chlorpropamide, tolbutamide, acetohexamide, tolazamide, glyburide, gliclazide, glynase, glimepiride, and glipizide); biguanides (such as metformin); a PPAR beta delta agonist; a ligand or agonist of PPAR gamma such as thiazolidinediones (such as ciglitazone, pioglitazone (see, e.g., U.S. Pat. No. 6,218,409), troglitazone, and rosiglitazone (see, e.g., U.S. Pat. No. 5,859,037)); PPAR alpha agonists such as clofibrate, gemfibrozil, fenofibrate, ciprofibrate, and bezafibrate; dehydroepiandrosterone (also referred to as DHEA or its conjugated sulphate ester, DHEA-SO4); antiglucocorticoids; TNFα inhibitors; α-glucosidase inhibitors (such as acarbose, miglitol, and voglibose); amylin and amylin derivatives (such as pramlintide, (see, also, U.S. Pat. Nos. 5,902,726; 5,124,314; 5,175,145 and 6,143,718.)); insulin secretogogues (such as repaglinide, gliquidone, and nateglinide (see, also, U.S. Pat. Nos. 6,251,856; 6,251,865; 6,221,633; 6,174,856)), and insulin.

X. Diagnosis of Diabetes

The present invention also provides methods of diagnosing diabetes or a predisposition of at least some of the pathologies of diabetes. Diagnosis can involve determination of a genotype of an individual (e.g., with SNPs) and comparison of the genotype with alleles known to have an association with the occurrence of diabetes or other SMAP-2-related disease. Alternatively, diagnosis also involves determining the level of SMAP-2 (protein or transcript) in a patient and then comparing the level to a baseline or range. Typically, the baseline value is representative of SMAP-2 in a healthy (e.g., lean) person. The level of a polypeptide in a lean individual can be a reading from a single individual, but is typically a statistically relevant average from a group of lean individuals. The level of a polypeptide in a lean individual can be represented by a value, for example in a computer program.

As discussed above, variation of levels (e.g., high levels) of SMAP-2 from the baseline range indicates that the patient is either diabetic or at risk of developing at least some of the pathologies of diabetes (e.g., pre-diabetic). In some embodiments, the level of SMAP-2 are measured by taking a blood, urine or tissue sample from a patient and measuring the amount of SMAP-2 in the sample using any number of detection methods, such as those discussed herein. For instance, fasting and fed blood or urine levels can be tested.

In some embodiments, the baseline level and the level in a lean sample from an individual, or at least two samples from the same individual differ by at least about 5%, 10%, 20%, 50%, 75%, 100%,150%, 200%,300%, 400%, 500%, 1000% or more. In some embodiments, the sample from the individual is greater by at least one of the above-listed percentages relative to the baseline level. In some embodiments, the sample from the individual is lower by at least one of the above-listed percentages relative to the baseline level.

In some embodiments, the level of SMAP-2 is used to monitor the effectiveness of antidiabetic therapies such as t hiazolidinediones, metformin, sulfonylureas and other standard therapies. In some embodiments the activity or expression of SMAP-2 will be measured prior to and after treatment of diabetic or pre-diabetic patients with antidiabetic therapies as a surrogate marker of clinical effectiveness. For example, the greater the reduction in SMAP-2 expression or activity indicates greater effectiveness.

Glucose tolerance tests can also be used to detect the effect of glucose levels on SMAP-2 levels. In glucose tolerance tests, the patient's ability to tolerate a standard oral glucose load is evaluated by assessing serum and urine specimens for glucose levels. Blood samples are taken before the glucose is ingested, glucose is given by mouth, and blood or urine glucose levels are tested at set intervals after glucose ingestion. SMAP-2 levels can also be determined. Such tests can also be performed in the presence and absence of a SMAP-2 modulator. Similarly, meal tolerance tests can also be used to detect the effect of food on SMAP-2 levels.

### EXAMPLES

The following examples are offered to illustrate, but not to limit the claimed invention.

Expression Levels of SMAP-2 in Human Tissues as Determined by Affymetrix Genechips™

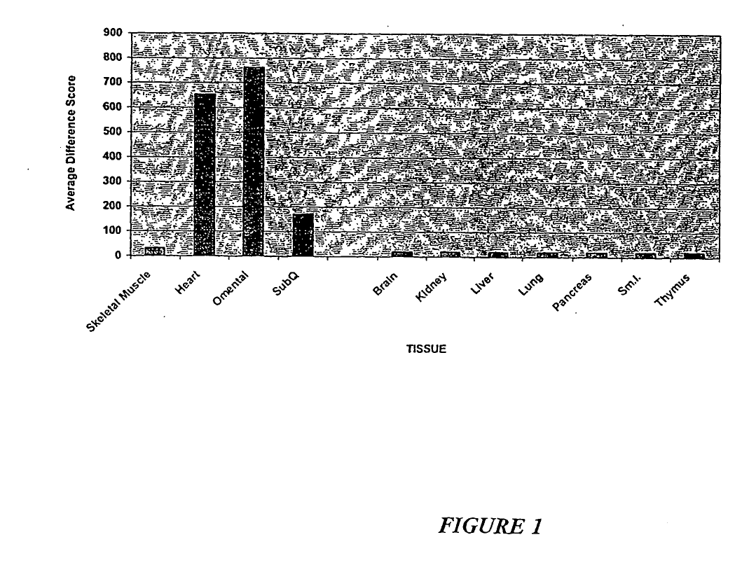

Genechip arrays were hybridized with cRNA prepared from a variety of human tissues including skeletal muscle, heart, omental and subcutaneous (SubQ) adipose, brain, kidney, liver, lung, small intestine (Sm.I), thymus and pancreas. FIG. 1 shows the average difference score obtained for SMAP-2 in these tissues. SMAP-2 was called present by the Affymetrix software in skeletal muscle, heart, omental and SubQ tissues and absent in all other tissues. The average difference scores for each of the tissues were: skeletal muscle-32, heart-652, omental adipose-762, and, subcutaneous adipose-171.

Expression of SMAP-2 in Insulin Responsive Tissue as Determined by Northern Blot

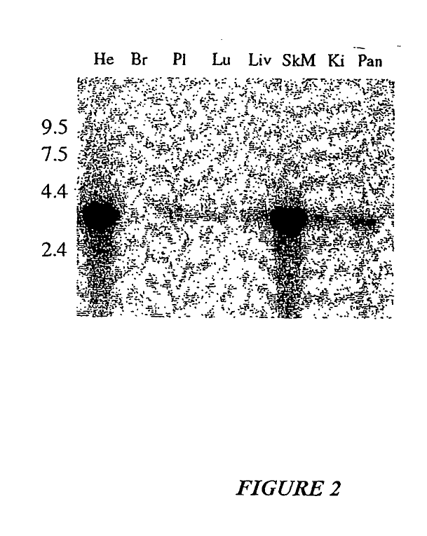

A multiple tissue northern blot derived from a variety of human tissues was hybridized with a SMAP2 probe. These tissues include heart (He), brain (Br), placenta (Pl), lung (Lu), liver (Liv), skeletal muscle (SkM), kidney (Ki) and pancreas (Pan). FIG. 2 demonstrates that SMAP-2 has a restricted human tissue expression pattern with highest levels been observed in heart and skeletal muscle.

Upregulation of SMAP-2 in Human Diabetic Skeletal Muscle as Determined by Affymetrix Genechips™

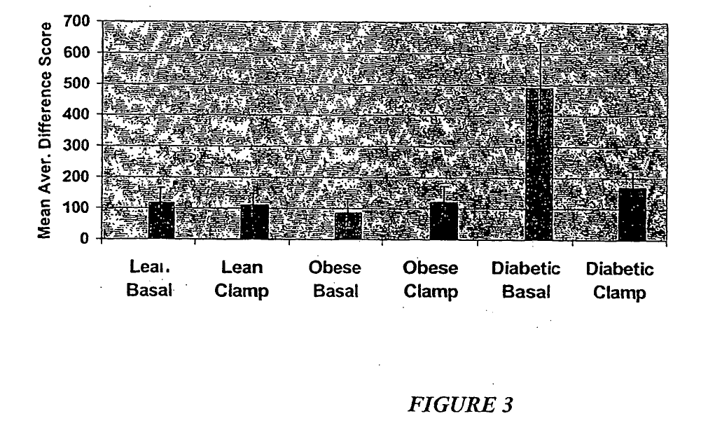

Genechip arrays were hybridized with cRNA prepared from lean (n=17), obese (n=16) and diabetic (n=19) skeletal muscle obtained after an overnight fast (Basal) or after a 5 hour hyperinsulinemic-euglycemic clamp (Clamp). As displayed in FIG. 3, SMAP-2 gene expression was upregulated 4.15 fold in diabetic subjects (DIABETIC) with statistical significance (students t-test p value of <0.028) when compared to the lean normal values (LEAN). The mean average difference scores±standard error were: Lean Basal 117.8±48.7; Lean Clamp 108.8±41.1; Obese Basal 85.7±43.8; Obese Clamp 122.1±51.3; Diabetic Basal 488.5±149.7 and Diabetic Clamp 167.3±54.1. Thus, SMAP-2 appears to be an upregulated gene in human diabetic skeletal muscle.

Reconfirmation of the Upregulation of SMAP-2 Expression Levels by Quantitative PCR in Human Diabetic Skeletal Muscle

Upregulation of SMAP-2 in human diabetic skeletal muscle was reconfirmed with quantitative PCR. PCR primers and Taqman Probes were designed using Perkin Elmer's Primer Express software (Version 1.5). Briefly, primers are chosen to produce an amplicon of 80-120 nucleotides in length. Specificity is obtained by using primers and probes that hybridize only to human SMAP-2.

| | |
|---|---|
| SMAP-2 probe: | |
| 6-fam- tgaccaacagaactggtctgcgtgc | (SEQ ID NO: 15) |
| SMAP-2 forward PCR primer: | |
| ggagtggaatgcccacaca | (SEQ ID NO: 16) |
| SMAP-2 reverse PCR primer: | |
| tgagggtggcagctggtta | (SEQ ID NO: 17) |

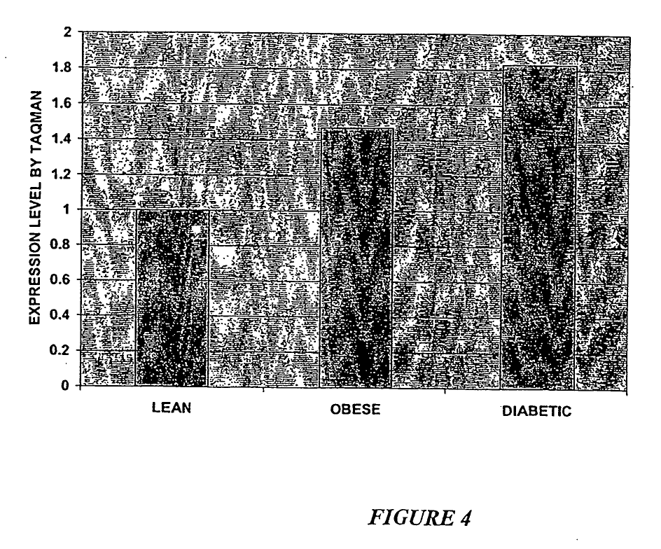

As displayed in FIG. 4, SMAP-2 levels were 1.47- and 1.83-fold upregulated in obese and diabetic basal skeletal muscle, respectively, when compared to lean. The relative expression level was determined by quantitative PCR.

Upregulation of SMOC-2 in a Mouse Genetic Model of Diabetes

Expression levels of SMOC2 was determined in db/db mice. PCR primers were designed using Perkin Elmer's Primer Express software (Version 1.5). Briefly, primers are chosen to produce an amplicon of 80-120 nucleotides in length. Specificity is obtained by using primers that hybridize only to mouse SMOC-2.

The SMOC-2 forward PCR primer is:

| | |
|---|---|
| TGGGTCACACAGGCACTAGCT | (SEQ ID NO: 18) |

The SMOC-2 reverse PCR primer is:

| | |
|---|---|
| TGGAGCCTAACAGCATAGTCTCAA | (SEQ ID NO: 19) |

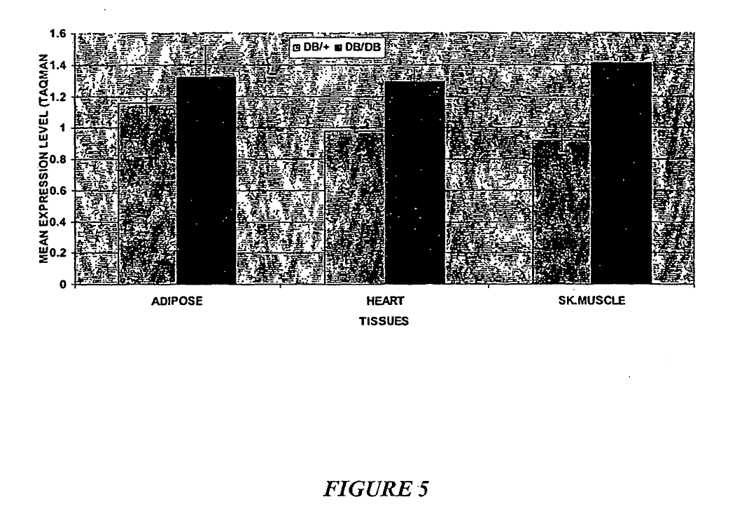

Db/db mice are a monogenic model for diabetes. SMOC-2 was 1.53-fold upregulated in skeletal muscle isolated from db/db mice when compared to normal heterozygotes db/+ with a P value of <0.001. See FIG. 5. The mean relative expression levels±standard error (n=5) as determined by quantitative PCR are 1.16±0.085 from epididymal adipose (ADIPOSE), 0.98±0.012 from heart (HEART) and 0.92±0.077 from skeletal muscle (SK. MUSCLE) prepared from normal db/+mice, and, 1.32±0.203 from epididymal adipose, 1.29±0.093 from heart and 1.41±0.04 from skeletal muscle prepared from diabetic db/db mice. No effect on SMOC-2 expression levels was observed in samples prepared from the adipose tissue.

Secretion of SMAP-2 from Mouse 3T3-L1 Adipocytes

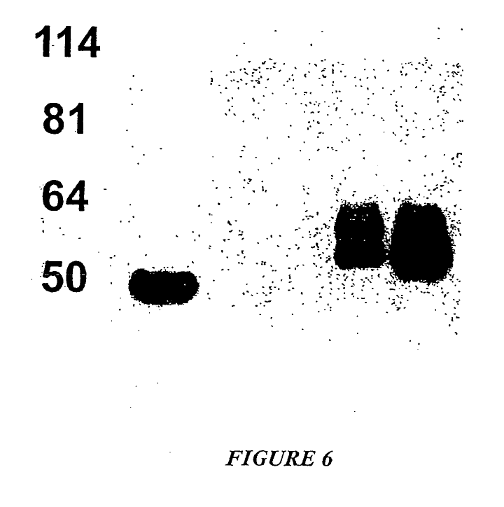

Secretion of SMAP-2 from mouse 3T3-L1 adipocytes was also analyzes 3T3-L1 adipocytes, 5-8 days post-differentiation, were removed from plates with trypsin, washed twice with PBS and resuspended in PBS at a concentration of 20×10⁶ cells per ml. Cells were electroporated with either 300 μg of control LacZ or wild-type SMAP-2 plasmid DNA and 50 μg DNA encoding eGFP-GLUT4. Following electroporation, the cells were replated either into 24 well plate or onto coverslips and incubated 36 hours in DMEM containing 10% FBS. After 36 hours the culture supernatant was separated from the cells. See, FIG. 6. One milliliter of supernatant (M) or 100 μg of total cellular extracts (C) were immunoprecipitated and immunoblotted with epitope tag anti-V5 antibodies. Standard molecular weight markers (KDa) are shown on the left of FIG. 6. The “++” label indicates an aliquot of recombinant SMAP-2 used as a positive control on the immunoblot.

Multiple bands were recognized by the anti-V5 antibodies. SMAP-2 was predicted to run at approximately 50 KDa, with proteins running higher probably representing glycosylated forms of SMAP-2.

Inhibition of GLUT4 Translocation by the Overexpression of Wild-Type SMAP-2 in Mouse 3T3-L1 Adipocytes

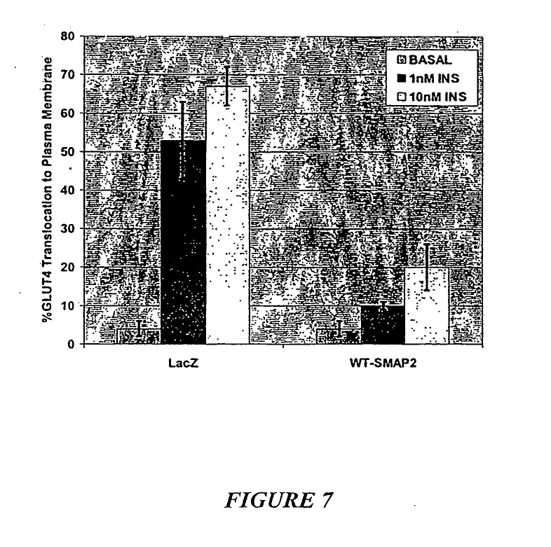

GLUT4 translocation was also analyzed in response to SMAP-2 expression. Mouse 3T3-L1 adipocytes were electroporated as described above. FIG. 7 displays representative GLUT4.eGFP translocation in control (basal) or after 1 nM or 10 nM insulin stimulation in 3T3-L1 adipocytes. Three separate experiments are shown and expressed as % of GLUT4.eGFP expressing cells showing translocation of GLUT4.eGFP to the plasma membrane in response to insulin and co-staining for the V5-epitope tagged SMAP-2. The % GLUT4.eGFP±SE was 4±2 (basal), 53±10 (1 nM insulin) and 67±5 (10 nM insulin) with LacZ and 4±2 (basal), 10±1 (1 nM insulin, pv<0.046) and 20±6 (10 nM insulin, pv<0.0003) in cells expressing wild-type SMAP-2.

In conclusion, expression of SMAP-2 potently inhibited GLUT4.eGFP translocation in response to insulin.

Overexpression of SMAP-2 Inhibits Insulin Stimulated Glucose Uptake in 3T3-L1 Adipocytes

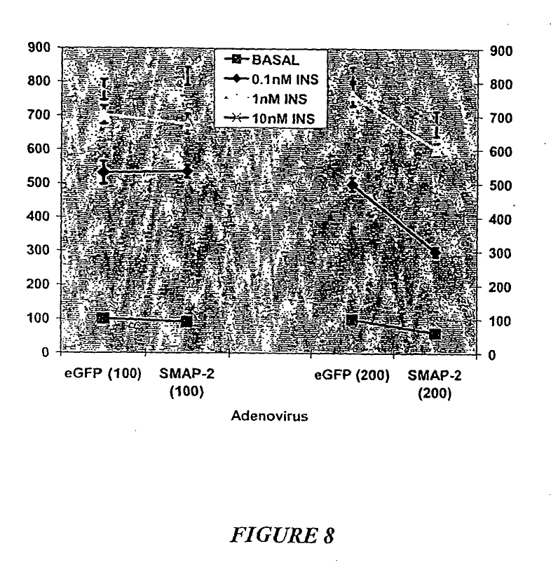

3T3-L1 adipocytes were infected with either control eGFP adenovirus (eGFP) or with wild-type SMAP-2 adenovirus (SMAP-2) at multiples of infection (MOIs) of either 100 or 200. After an overnight incubation, the cells were stimulated with insulin (INS) at basal 0.1, 1, or 10 nM for 30 minutes. The adipocytes were assayed acutely for glucose transport activity by the measurement of [3H]2-deoxy-glucose (2-DOG) uptake. Each assay was performed in triplicate. The results are displayed in FIG. 8 and Table 1 (below). The data are presented as the fold change±SE in 2-DOG uptake (n=3) when compared to the eGFP basal which has been set to 100.

The raw counts are as follows: eGFP (100) Basal 5049±166; eGFP (100)+0.1 nM INS 26835±1708; eGFP (100)+1 nM INS 35453±1350; eGFP (100)+10 nM INS 39195±1353; SMAP-2 (100) Basal 4655±90; SMAP-2 (100)+0.1 nM INS 27041±927; SMAP-2 (100)+1 nM INS 34150±1502; SMAP-2 (100)+10 nM INS 41293±1353.eGFP (200) Basal 3680±86; eGFP (200)+0.1 nM INS 18353±738; eGFP (200)+1 nM INS 28096±1213; eGFP (200)+10 nM INS 30360±740; SMAP-2 (200) Basal 2225±25; SMAP-2 (200)+0.1 nM INS 11013±518; SMAP-2 (200)+1 nM INS 22151±692; SMAP-2 (200)+10 nM INS 24931±1361.

TABLE 1
Fold changes for the insulin induced increases in glucose uptake
over control eGFP adenovirus as determined from glucose uptake.
The Students t-test was used to compare groups.

| | | | +0.1 nM | | +1 nM | | +10 nM | |
|---|---|---|---|---|---|---|---|---|
| | BASAL | | INS | | INS | | INS | |
| | FC | pvalue | FC | p-value | FC | p-value | FC | pvalue |
| SMAP-2 | 0.92 | 0.126 | 1.01 | 0.922 | 0.96 | 0.554 | 1.05 | 0.363 |
| (100) | | | | | | | | |
| SMAP-2 | 0.61 | 0.002 | 0.60 | 0.002 | 0.79 | 0.02 | 0.82 | 0.037 |
| (200) | | | | | | | | |

Values of p ≦ 0.05 were considered significant.

A SMAP-2 Promoter-Based Assay for High Throughput Screening

An example of a high throughput screening assay to screen for compounds that may modulate the expression of SMAP2 was established. Bioinformatics analysis of the human SMAP2 genomic sequence encompassing 220 kb on chromosome 6 suggested that the transcription start site is likely to be about 320 bp upstream of the start codon. Focusing on the 1.2 kb genomic region upstream of the start codon of SMAP2 promoter, there is a 55% sequence identity between the human and mouse, suggesting that this region may harbor essential transcriptional regulatory elements to function as a promoter. We cloned this 1.2 kb human SMAP2 promoter region upstream of a pGL3 luciferase reporter vector, thus, rendering the expression of luciferase under the influence of SMAP2 promoter in a cell-based assay.

Either C2C12, L6, CHO K1 or 293T cell lines were transiently transfected in a 96 well plate format with a control (pGL3 background) or a luciferase reporter under the control of SMAP2 promoter (pGL3 SMAP2 promoter) and an internal control pcDNA4 His-max-lac Z driving the expression of β-galactosidase. Twenty-four hours after the transfection, cells were processed for both luciferase and β-galactosidase assays. The assays were performed in triplicate. The relative luciferase activity (luciferase activity/β-galactosidase activity) of the SMAP2 reporter in these cell lines are shown in the table below. Compared to the control pGL3 background, the SMAP2 promoter studied contains strong promoter activity in different cell types tested. The SMAP2 promoter is apparently also subjected to cell type specific regulation with the strongest level of activity in L6 cells.

| | Relative Luciferase Activity | | Relative Luciferase Activity | |
|---|---|---|---|---|
| | pGL3 background | | pGL3 SMAP2 promoter | |
| Cell Type | Avg. | STD. | Avg | STD. |
| C2C12 | 4 | 1 | 104 | 17 |
| L6 | 28 | 2 | 808 | 71 |
| CHO K1 | 12 | 1 | 72 | 10 |
| 293 | 20 | 3 | 61 | 6 |

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A method for identifying an agent for treating a diabetic or pre-diabetic individual, the method comprising the steps of:
(i) contacting an agent with a solution comprising a polypeptide encoded by a nucleic acid that hybridizes under stringent conditions to a nucleic acid encoding SEQ ID NO:2, SEQ ID NO:4, SEQ ID NO:6 or SEQ ID NO:9;
(ii) selecting an agent that decreases the expression or activity of the polypeptide or that binds to the polypeptide; and
(iii) testing the selected agent for the ability to modulate insulin sensitivity in a cell thereby identifying an agent capable of modulating insulin sensitivity in a cell.

2. The method of claim 1, comprising selecting an agent that decreases expression of the polypeptide.

3. The method of claim 1, comprising selecting an agent that decreases activity of the polypeptide.

4. The method of claim 1, comprising selecting an agent that binds to the polypeptide.

5. The method of claim 1, wherein the contacting step is performed in vitro.

6. The method of claim 1, wherein the testing step comprises administering the agent to an animal and testing the animal for modulated insulin sensitivity.

7. The method of claim 6, wherein the animal exhibits insulin resistance prior to administration.

8. The method of claim 1, wherein the testing step comprises contacting a cell expressing the polypeptide with the agent and testing the cell for modulated insulin sensitivity.

9. The method of claim 8, wherein the testing comprises selecting an agent that increases glucose uptake in the cell compared to a cell not contacted with the agent.

10. The method of claim 8, wherein the testing comprises selecting an agent that increases GLUT4 translocation in the cell compared to a cell not contacted with the agent.

11. The method of claim 1, wherein the cell is an adipocyte, heart cell or skeletal muscle cell.

12. The method of claim 1, wherein the cell is a human cell.

13. The method of claim 1, wherein the amino acid sequence comprises SEQ ID NO:2.

14. The method of claim 1, wherein the amino acid sequence comprises SEQ ID NO:4.

15. The method of claim 1, wherein the amino acid sequence comprises SEQ ID NO:6.

16. The method of claim 1, wherein the amino acid sequence comprises SEQ ID NO:9.

17. The method of claim 1, wherein the agent is an antibody.

18. The method of claim 17, wherein the antibody is a monoclonal antibody.

19. The method of claim 1, wherein the agent is an antisense polynucleotide.

20. The method of claim 1, wherein the testing step comprises testing glucose uptake of the cell.

21. The method of claim 1, wherein the testing step comprises testing GLUT4 translocation in the cell.

22. A method of treating a diabetic or pre-diabetic animal, the method comprising administering to the animal a therapeutically effective amount of an agent identified by the method of claim 1.

23. The method of claim 22, wherein the agent is an antibody.

24. The method of claim 23, wherein the antibody is a monoclonal antibody.

25. The method of claim 22, wherein the agent is an antisense polynucleotide.

26. The method of claim 23, wherein animal is a human.

27. A method of treating a diabetic or pre-diabetic animal, the method comprising administering to the animal a therapeutically effective amount of an agent that specifically binds to SEQ ID NO:2.

28. The method of claim 27, wherein the agent is a polypeptide.

29. The method of claim 27, wherein the agent is an antibody.

30. The method of claim 29, wherein the antibody is a monoclonal antibody.

31. The method of claim 27, wherein the agent is an antisense polynucleotide.

32. The method of claim 27, wherein the animal is a human.

33. A method of diagnosing a diabetic or pre-diabetic individual, the method comprising,
detecting in a sample from the individual the level of a polypeptide encoded by a nucleic acid that hybridizes under stringent conditions to a nucleic acid encoding SEQ ID NO:2,
wherein an increased level of the polypeptide in the sample compared to a level of the polypeptide in a lean person or a previous sample from the individual indicates that the individual is diabetic or pre-diabetic.

34. The method of claim 27, wherein the individual has Type 2 diabetes.

35. The method of claim 27, wherein the individual is pre-diabetic.

36. The method of claim 33, wherein the sample is a blood, urine or tissue sample.

37. The method of claim 33, wherein the detecting step comprises contacting the sample with an antibody that specifically binds to the polypeptide.

38. A method of diagnosing an individual who has Type 2 diabetes or is pre-diabetic, the method comprising,
detecting in a sample from the individual the level of a polynucleotide that hybridizes under stringent conditions to a nucleic acid encoding SEQ ID NO:2,
wherein an increased level of the polynucleotide in the sample compared to a level of the polynucleotide in a lean person or a previous sample from the individual indicates that the individual is diabetic or pre-diabetic.

39. The method of claim 38, wherein the detecting step comprises quantifying mRNA that hybridizes under stringent conditions to a nucleic acid encoding SEQ ID NO:2.

40. The method of claim 39, wherein the mRNA is reverse transcribed and amplified in a polymerase chain reaction.

41. The method of claim 38, wherein the sample is a blood, urine or tissue sample.

42. An isolated nucleic acid comprising a polynucleotide encoding a polypeptide comprising:
amino acids 351 to 379 of SEQ ID NO:2;
amino acids 388 to 416 of SEQ ID NO:2;
amino acids 40 to 84 of SEQ ID NO:2;
amino acids 90 to 153 of SEQ ID NO:2; and/or
amino acids 216 to 281 of SEQ ID NO:2.

43. The nucleic acid of claim 42, wherein the polypeptide comprises SEQ ID NO:2.

44. The nucleic acid of claim 42, wherein the polynucleotide is SEQ D NO:1.

45. An expression cassette comprising a heterologous promoter operably linked to the nucleic acid of claim 42.

46. A host cell transfected with the nucleic acid of claim 42.

47. The host cell of claim 46, wherein the host cell is a human cell.

48. The host cell of claim 46, wherein the host cell is a bacteria.

49. An isolated polypeptide comprising:
amino acids 351 to 379 of SEQ ID NO:2;
amino acids 388 to 416 of SEQ ID NO:2;
amino acids 40 to 84 of SEQ ID NO:2;
amino acids 90 to 153 of SEQ ID NO:2; and/or
amino acids 216 to 281 of SEQ ID NO:2.

50. The polypeptide of claim 49, wherein the polypeptide comprises SEQ ID NO:2.

51. An expression cassette comprising a promoter operably linked to a heterologous polynucleotide, wherein the promoter comprises SEQ ID NO:10 or SEQ ID NO:11.

52. The expression cassette of claim 51, wherein the polynucleotide encodes a polypeptide.

53. The expression cassette of claim 51, wherein the polypeptide is a reporter gene product.

54. A cell comprising the expression cassette of claim 51.

55. A method of expressing a polynucleotide in a muscle cell, the method comprising introducing an expression cassette comprising a promoter operably linked to a heterologous polynucleotide, wherein the promoter comprises SEQ ID NO:10 or SEQ ID NO:11.


SEQUENCE LISTING (20 sequences)

SEQ ID NO: 1 - DNA, 1341 bp, Homo sapiens
  human smooth muscle associated protein-2 (SMAP-2) open reading frame
atgctgctcccccagctctgctggctgccgctgctcgctgggctgctcccgccggtgcccgcgcagaagttctcggtgctcacgtttttgagagtggatcaagataaagacaaggattgtagcttggactgtgcgggttcgccccagaaacctctctgcgcatctgacggaaggaccttcctttcccgttgtgaatttcaacgtgccaagtgcaaagatccccagctagagattgcatatcgaggaaactgcaaagacgtgtccaggtgtgtggccgaaaggaagtatacccaggagcaagcccggaaggagtttcagcaagtgttcattcctgagtgcaatgacgacggcacctacagtcaggtccagtgtcacagctacacgggatactgctggtgcgtcacgcccaacgggaggcccatcagcggcactgccgtggcccacaagacgccccggtgcccgggttccgtaaatgaaaagttaccccaacgcgaaggcacaggaaaaacagatgatgccgcagctccagcgttggagactcagcctcaaggagatgaagaagatattgcatcacgttaccctaccctttggactgaacaggttaaaagtcggcagaacaaaaccaataagaattcagtgtcatcctgtgaccaagagcaccagtctgccctggaggaagccaagcagcccaagaacgacaatgtggtgatccctgagtgtgcgcacggcggcctctacaagccagtgcagtgccacccctccacggggtactgctggtgcgtcctggtggacacggggcgccccattcccggcacatccacaaggtacgagcagccgaaatgtgacaacacggccagggcccacccagccaaagcccgggacctgtacaagggccgccagctacaaggttgtccgggtgccaaaaagcatgagtttctgaccagcgttctggacgcgctgtccacggacatggtccacgccgcctccgacccctcctcctcgtcaggcaggctctcagaacccgaccccagccataccctagaggagcgggtggtgcactggtacttcaaactactggataaaaactccagtggagacatcggcaaaaaggaaatcaaacccttcaagaggttccttcgcaaaaaatcaaagcccaaaaaatgtgtgaagaagtttgttgaatactgtgacgtgaataatgacaaatccatctccgtacaagaactgatgggctgcctgggcgtggcgaaagaggacggcaaagcggacaccaagaaacgccacacccccagaggtcatgctgaaagtacgtctaatagacagccaaggaaacaaggataa

SEQ ID NO: 2 - protein, 446 aa, Homo sapiens
  human smooth muscle associated protein-2 (SMAP-2)
MLLPQLCWLPLLAGLLPPVPAQKFSVLTFLRVDQDKDKDCSLDCAGSPQKPLCASDGRTFLSRCEFQRAKCKDPQLEIAYRGNCKDVSRCVAERKYTQEQARKEFQQVFIPECNDDGTYSQVQCHSYTGYCWCVTPNGRPISGTAVAHKTPRCPGSVNEKLPQREGTGKTDDAAAPALETQPQGDEEDIASRYPTLWTEQVKSRQNKTNKNSVSSCDQEHQSALEEAKQPKNDNVVIPECAHGGLYKPVQCHPSTGYCWCVLVDTGRPIPGTSTRYEQPKCDNTARAHPAKARDLYKGRQLQGCPGAKKHEFLTSVLDALSTDMVHAASDPSSSSGRLSEPDPSHTLEERVVHWYFKLLDKNSSGDIGKKEIKPFKRFLRKKSKPKKCVKKFVEYCDVNNDKSISVQELMGCLGVAKEDGKADTKKRHTPRGHAESTSNRQPRKQG

SEQ ID NO: 3 - DNA, 2914 bp, Homo sapiens
  human smooth muscle associated protein-2 (SMAP-2) cDNA
ctcccgccacctccgccaccatgctgctcccccagctctgctggctgccgctgctcgctgggctgctcccgccggtgcccgctcagaagttctcggcgctcacgtttttgagagtggatcaagataaagacaaggattgtagcttggactgtgcgggttcgccccagaaacctctctgcgcatctgacggaaggaccttcctttcccgttgtgaatttcaacgtgccaagtgcaaagatccccagctagagattgcatatcgaggaaactgcaaagacgtgtccaggtgtgtggccgaaaggaagtatacccaggagcaagcccggaaggagtttcagcaagtgttcattcctgagtgcaatgacgacggcacctacagtcaggtccagtgtcacagctacacgggatactgctggtgcgtcacgcccaacgggaggcccatcagcggcactgccgtggcccacaagacgccccggtgcccgggttccgtaaatgaaaagttaccccaacgcgaaggcacaggaaaaacagatgatgccgcagctccagcgttggagactcagcctcaaggagatgaagaagatattgcatcacgttaccctaccctttggactgaacaggttaaaagtcggcagaacaaaaccaataagaatccagtgtcatcctgtgaccaagagcaccagtctgccctggaggaagccaagcagcccaagaacgacaatgtggtgatccctgagtgtgcgcacggcggcctctacaagccagtgcagtgccacccctccacggggtactgctggtgcgtcctggtggacacggggcgccccattcccggcacatccacaaggtacgagcagccgaaatgtgacaacacggccagggcccacccagccaaagcccgggacctgtacaagggccgccagctacaaggttgtccgggtgccaaaaagcatgagtttctgaccagcgttctggacgcgctgtccacggacatggtccacgccgcctccgacccctcctcctcgtcaggcaggctctcagaacccgaccccagccataccctagaggagcgggtggtgcactggtacttcaaactactggataaaaactccagcggagacatcggcaaaaaggaaatcaaacccttcaagaggttccttcgcaaaaaatcaaagcccaaaaaatgtgtgaagaagtttgttgaatactgtgacgtgaataatgacaaatccatctccgtacaagaactgatgggctgcctgggcgtggcgaaagaggacggcaaagcggacaccaagaaacgccacacccccagaggtcatgctgaaagtacgtctaatagacagccaaggaaacaaggataaatggctcataccccgaaggcagttcctagacacatgggaaatttccctcaccaaagagcaattaagaaaacaaaaacagaaacacatagtatttgcactttgtactttaaatgtaaattcactttgtagaaatgagctatttaaacagactgttttaatctgtgaaaatggagagctggcttcagaaaattaatcacatacaatgtatgtgtcctcttttgaccttggaaatctgtatgtggtggagaagtatttgaatgcatttaggcttaatttcttcgccttccacatgttaacagtagagctctatgcactccggctgcaatcgtatggctttctctaacccctgcagtcacttccagatgcctgtgcttacagcattgtggaatcatgttggaagctccacatgtccatggaagtttgtgatgtacggccgaccctacaggcagttaacatgcatgggctggtttgtttcttgggattttcttttagtttgtcttgttttgctttccagagatcttgctcatacaatgaatcacgcaaccactaaagctatccagttaagtgcaggtagttcccctggaggaaataatattttcaaactgtcgttggtgtgatactttggctcaaaggatctttgcttttccattttaagcttctgttttgagttttgccctggggcttgaatgagtcccagagagtcgttccgatggtgggaggctgcctaggaggcagtaaatccagtcacagtgcctgggaggggcccatccttccaaaatgtaaatccagtcgcggtgtgaccgagctggctaacaggcttgtctgcctggttttcctcctacacatggacattattctcctgatcctcctacctggtccaccccagggctaccggaaggtaaaatcttcacctgaaccaattatgagcagtctccttactgaaggtacagccggatacgtggtgcccccggggctggtgttggcagccggggggaggtgcctgagggtccccacggttcctttctgcttttctgaatgcatcaagggtacgagaacttgccaatgggaaattcatccgagtggcactggcagagaaggataggagtggaatgcccacacagtgaccaacagaactggtctgcgtgcataaccagctgccaccctcaggcctgggccccagagctcagggcacccagtgtcttaaggaaccatttggaggacagtctgagagcaggaacttcaagctgtgattctatctcggctcagacttttggttggaaaaagatcttcatggccccaaatcccctgagacatgccttgtagaatgattttgtgatgttgtgatgcttgtggagcatcgcgtaaggcttcttgcttatttaaactgtgcaaggtaaaaatcaagcctttggagccacagaaccagctcaagtacatgccaatgttgtttaagaaacagttatgatcctaaactttttggataatcttttatatttctgacctttgaatttaatcattgttcttagattaaaataaaatatgctattgaaact

SEQ ID NO: 4 - protein, 446 aa, Homo sapiens [sequence omitted]

SEQ ID NO: 5 - DNA, 2947 bp, Homo sapiens
  human smooth muscle associated protein-2B (SMAP-2B) cDNA
ctcccgccacctccgccaccatgctgctcccccagctctgctggctgccgctgctcgctgggctgctcccgccggtgcccgctcagaagttctcggcgctcacgtttttgagagtggatcaagataaagacaaggattgtagcttggactgtgcgggttcgccccagaaacctctctgcgcatctgacggaaggaccttcctttcccgttgtgaatttcaacgtgccaagtgcaaagatccccagctagagattgcatatcgaggaaactgcaaagacgtgtccaggtgtgtggccgaaaggaagtatacccaggagcaagcccggaaggagtttcagcaagtgttcattcctgagtgcaatgacgacggcacctacagtcaggtccagtgtcacagctacacgggatactgctggtgcgtcacgcccaacgggaggcccatcagcggcactgccgtggcccacaagacgccccggtgcccgggttccgtaaatgaaaagttaccccaacgcgaaggcacaggaaaaacagtctccttgcaaatcttttccgttctgaattcagatgatgccgcagctccagcgttggagactcagcctcaaggagatgaagaagatattgcatcacgttaccctaccctttggactgaacaggttaaaagtcggcagaacaaaaccaataagaattcagtgtcatcctgtgaccaagagcaccagtctgccctggaggaagccaagcagcccaagaacgacaatgtggtgatccctgagtgtgcgcacggcggcctctacaagccagtgcagtgccacccctccacggggtactgctggtgcgtcctggtggacacggggcgccccattcccggcacatccacaaggtacgagcagccgaaatgtgacaacacggccagggcccacccagccaaagcccgggacctgtacaagggccgccagctacaaggttgtccgggtgccaaaaagcatgagtttctgaccagcgttctggacgcgctgtccacggacatggtccacgccgcctccgacccctcctcctcgtcaggcaggctctcagaacccgaccccagccataccctagaggagcgggtggtgcactggtacttcaaactactggataaaaactccagcggagacatcggcaaaaaggaaatcaaacccttcaagaggttccttcgcaaaaaatcaaagcccaaaaaatgtgtgaagaagtttgttgaatactgtgacgtgaataatgacaaatccatctccgtacaagaactgatgggctgcctgggcgtggcgaaagaggacggcaaagcggacaccaagaaacgccacacccccagaggtcatgctgaaagtacgtctaatagacagccaaggaaacaaggataaatggctcataccccgaaggcagttcctagacacatgggaaatttccctcaccaaagagcaattaagaaaacaaaaacagaaacacatagtatttgcactttgtactttaaatgtaaattcactttgtagaaatgagctatttaaacagactgttttaatctgtgaaaatggagagctggcttcagaaaattaatcacatacaatgtatgtgtcctcttttgaccttggaaatctgtatgtggtggagaagtatttgaatgcatttaggcttaatttcttcgccttccacatgttaacagtagagctctatgcactccggctgcaatcgtatggctttctctaacccctgcagtcacttccagatgcctgtgcttacagcattgtggaatcatgttggaagctccacatgtccatggaagtttgtgatgtacggccgaccctacaggcagttaacatgcatgggctggtttgtttcttgggattttcttttagtttgtcttgttttgctttccagagatcttgctcatacaatgaatcacgcaaccactaaagctatccagttaagtgcaggtagttcccctggaggaaataatattttcaaactgtcgttggtgtgatactttggctcaaaggatctttgcttttccattttaagcttctgttttgagttttgccctggggcttgaatgagtcccagagagtcgttccgatggtgggaggctgcctaggaggcagtaaatccagtcacagtgcctgggaggggcccatccttccaaaatgtaaatccagtcgcggtgtgaccgagctggctaacaggcttgtctgcctggttttcctcctacacatggacattattctcctgatcctcctacctggtccaccccagggctaccggaaggtaaaatcttcacctgaaccaattatgagcagtctccttactgaaggtacagccggatacgtggtgcccccggggctggtgttggcagccggggggaggtgcctgagggtccccacggttcctttctgcttttctgaatgcatcaagggtacgagaacttgccaatgggaaattcatccgagtggcactggcagagaaggataggagtggaatgcccacacagtgaccaacagaactggtctgcgtgcataaccagctgccaccctcaggcctgggccccagagctcagggcacccagtgtcttaaggaaccatttggaggacagtctgagagcaggaacttcaagctgtgattctatctcggctcagacttttggttggaaaaagatcttcatggccccaaatcccctgagacatgccttgtagaatgattttgtgatgttgtgatgcttgtggagcatcgcgtaaggcttcttgcttatttaaactgtgcaaggtaaaaatcaagcctttggagccacagaaccagctcaagtacatgccaatgttgtttaagaaacagttatgatcctaaactttttggataatcttttatatttctgacctttgaatttaatcattgttcttagattaaaataaaatatgctattgaaact

SEQ ID NO: 6 - protein, 457 aa, Homo sapiens
  human smooth muscle associated protein-2 (SMAP-2)
MLLPQLCWLPLLAGLLPPVPAQKFSALTFLRVDQDKDKDCSLDCAGSPQKPLCASDGRTFLSRCEFQRAKCKDPQLEIAYRGNCKDVSRCVAERKYTQEQARKEFQQVFIPECNDDGTYSQVQCHSYTGYCWCVTPNGRPISGTAVAHKTPRCPGSVNEKLPQREGTGKTVSLQIFSVLNSDDAAAPALETQPQGDEEDIASRYPTLWTEQVKSRQNKTNKNSVSSCDQEHQSALEEAKQPKNDNVVIPECAHGGLYKPVQCHPSTGYCWCVLVDTGRPIPGTSTRYEQPKCDNTARAHPAKARDLYKGRQLQGCPGAKKHEFLTSVLDALSTDMVHAASDPSSSSGRLSEPDPSHTLEERVVHWYFKLLDKNSSGDIGKKEIKPFKRFLRKKSKPKKCVKKFVEYCDVNNDKSISVQELMGCLGVAKEDGKADTKKRHTPRGHAESTSNRQPRKQG

SEQ ID NO: 7 - DNA, 3111 bp, Homo sapiens
  atherosclerosis-associated gene
cgagggcggacgcaaagaacgcggaggacctctgggtgcctgcnggggagctgctccagccgggccgccgggagcggtggggagagcatcgcgcagccgcccctccacgcgcccgcccagccgcgttcgcccactgggctctcccggctgcagtgccagggcgcaggacgcggccgatctcccgctcccgccacctccgccaccatgctgctcccccagctctgctggctgccgctgctcgctgggctgctcccgccggtgcccgctcagaagttctcggcgctcacgtttttgagagtggatcaagataaagacaaggattgtagcttggactgtgcgggttcgccccagaaacctctctgcgcatctgacggaaggaccttcctttcccgttgtgaatttcaacgtgccaagtgcaaagatccccagctagagattgcatatcgaggaaactgcaaagacgtgtccaggtgtgtggccgaaaggaagtatacccaggagcaagcccggaaggagtttcagcaagtgttcattcctgagtgcaatgacgacggcacctacagtcaggtccagtgtcacagctacacgggatactgctggtgcgtcacgcccaacgggaggcccatcagcggcactgccgtggcccacaagacgccccggtgcccgggttccgtaaatgaaaagttaccccaacgcgaaggcacaggaaaaacagatgatgccgcagctccagcgttggagactcagcctcaaggagatgaagaagatattgcatcacgttaccctaccctttggactgaacaggttaaaagtcggcagaacaaaaccaataagaattcagtgtcatcctgtgaccaagagcaccagtctgccctggaggaagccaagcagcccaagaacgacaatgtggtgatccctgagtgtgcgcacggcggcctctacaagccagtgcagtgccacccctccacggggtactgctggtgcgtcctggtggacacggggcgccccattcccggcacatccacaaggtacgagcagccgaaatgtgacaacacgggccagggcccacccagccaaagcccgggacctgtacaagggccgccagctacaaggttgtccgggtgccaaaaagcatgagtttctgaccagcgttctggacgcgctgtccacggacatggtccacgccgcctccgacccctcctcctcgtcaggcaggctctcagaacccgaccccagccataccctagaggagcgggtggtgcactggtacttcaaactactggataaaaactccagtggagacatcggcaaaaaggaaatcaaacccttcaagaggttccttcgcaaaaaatcaaagcccaaaaaatgtgtgaagaagtttgttgaatactgtgacgtgaataatgacaaatccatctccgtacaagaactgatgggctgcctgggcgtggcgaaagaggacggcaaagcggacaccaagaaacgccacacccccagaggtcatgctgaaagtacgtctaatagacagccaaggaaacaaggataaatggctcataccccgaaggcagttcctagacacatgggaaatttccctcaccaaagagcaattaagaaaacaaaaacagaaacacatagtatttgcactttgtactttaaatgtaaattcactttgtagaaatgagctatttaaacagactgttttaatctgtgaaaatggagagctggcttcagaaaattaatcacataccaatgtatgtgtcctcttttgaccttggaaatctgtatgtggtggagaagtatttgaatgcatttaggcttaatttcttcgccttccacatgttaacagtagagctctatgcactccggctgcaatcgtatggctttctctaacccctgcagtcacttccagatgcctgtgcttacagcattgtggaatcatgttggaagctccacatgtccatggaagtttgtgatgtacggccgaccctacaggcagttaacatgcatgggctggtttgtttcttgggattttctgttagtttgtcttgttttgctttccagagatcttgctcatacaatgaatcacgcaaccactaaagctatccagttaagtgcaggtagttcccctggaggaaataatattttcaaactgtcgttggtgtgatactttggctcaaaggatctttgcttttccattttaagcttctgttttgagttttgccctggggcttgaatgagtcccagagagtcgttcggatggtgggaggctgcctaggaggcagtaaatccagttcacagtgcctgggaggggcccatccttccaaaatgtaaatccagttcgcggtgtgaccgagctgggctaacaggcttgtctgcctggttttcctacctacacgtggacattattctcctgatcctcctacctggttccaccccagggctaccggaaggtaaaatcttcacctgaaccaattatgagcagtctccttactgaaggtacagccggatacgtggtgcccccggggctggtgttggcagccggggggaggtgcctgagggtccccacggttcctttctgcttttctgaatgcatcaagggtacgagaacttgccaatgggaaattcatccgagtggcactggcagagaaggataggagtggaatgcccacacagtgaccaacagaactggtctgcgtgcataaccagctgccaccctcaggcctgggccccagagctcagggcacccagtgtcttaaggaaccatttggaggacagtctgagagcaggaacttcaagctgtgattctatctcggctcagacttttggttggaaaaagatcttcatggccccaaatcccctgagacatgccttgtagaatgattttgtgatgttgtgatgcttgtggagcatcgcgtaaggcttcttgcttatttaaactgtgcaaggtaaaaatcaagcctttggagccacagaaccagctcaagtacatgccaatgttgtttaagaaacagttatgatcctaaactttttggataatcttttatatttctgacctttgaatttaatcattgttcttagattaaaataaaatatgctattgaaactaaaaaa

SEQ ID NO: 8 - DNA, 2774 bp, Mus musculus
  mouse ortholog of human smooth muscle associated protein-2 (SMAP-2), secreted modular calcium-binding protein-2 (SMOC-2) cDNA
gcggcagttcggggagcgccggccagagcgcacggaggggccgtgcggtctccaccagcggccataggaccgcgaagcagttctagcccgctcgcccgcacgttcgcacaccggatcttcgccgagtgccagggcgcagcgcgtggggcgtctgcctcgcttggtcccctccagcgtcaccatgctgccgccacagctgtgctggctgccgctgctcgctgccttgctgccgccggtgcccgcgcagaagttctcagcgctcacgttcttgagagtcgatcaagacaaagacagagactgcagcctggactgcccaagctcccctcagaagccactctgtgcctcggacgggaggaccttcctgtcccgatgtgagttccagcgggccaagtgcaaagatccacagctggagatcgctcaccgtgggaattgcaaagatgtgtccaggtgtgtggctgagaggaagtatacccaggagcaggcccggaaggagttccagcaagtgttcattccagaatgcaatgatgacggcacctacagtcaggtccagtgtcacagctacacaggatactgttggtgtgttacaccaaatggaagacccatcagtggcactgctgtggcccacaagacacccaggtgccccggttcaataaatgaaaaggtgccgcagcgggaaggagcagggaaagcagatgatgctgcagccccagcattggagactcagccccaaggagatgaagaagatattgcctcacgctaccctacactctggaccgagcaagttaagagtcggcagaacaagaccaataaaaattcagcatcctcctgtgatcaggagcatcagtcagctcttgaggaagccaagcagcccaagaatgacaatgtagtgatccccgagtgtgcacatggtggtctctacaagccagtgcaatgccatccatccaccggatactgctggtgtgtgctagtggacactggacggcccattcctgggacctccacaaggtatgagcaacctaagtgtgacaacacagcccgagctcacccagcgaaggcccgggacctgtacaagaacaggccactgcagggttgtcctggtgccaaaaagcacgagtttctgacaagtgtcctggatgcgctctccacagacatggttcatgctgtctctgacccctcttcctcttctggcaggctgtcagagccagaccccagccacaccctggaggagagggttgtacattggtacttcaagctgcttgataagaactctagtggagacattggcaagaaggagatcaaaccctttaagaggttcctgcgaaagaaatccaagcccaaaaagtgtgtgaagaagtttgtggagtactgcgacatgaacaatgacaagtccatcaccgtgcaggagctcatgggctgcttgggtgtcaccagagaggagggtaaagccaacaccaggaagcgccacacccccagaggaaatgctgaaagtacttctaatagacagcccaggaaacaaggatgaacggctgactactcaagacagttcctagacatgtgggaattttccctcaccaaagagcaattaaaaacaaaaaacatatagtatttgcactttgtactttaaatgtaaattcactttgtagaaatgagatatttaaacggactgttgtgatctgtgaaaacggaaggctggcttcgggaaattaatcacatacaatgtatgtgtccttgtttgaccttcaagatctgtgctggtggggtgggttttaaatgcatttcaacttcacttcctcgtccctctgtggagggcctggtttattcccttgccctttcctctgcctgcagtctccgttggcttgctctccgtgctcttgatgccagccagagcctaccgtatccttagaagcttgcagtggttgcctgtttcataaagttttgctctctagagatgtcactcacaccatgggtcacacaggcactagctcaggcatagccttaacttccagtagcccctgcttgagactatgctgttaggctccagttttagcttctgcttgagccaaccctactgttaaatccccggggagctgaatggacaagcagccagagagtcctctttcagagagcacatctaactctggcctgccgactccatctcattggcccaggaaggatccagtctacctgcctcacttctgagtgcctgggccatcctgctagggtccactggaaaggaaaaatgccttctacagagatcatggaccacatcctgaggaaggcacagctagactacatcgtcccaagagtgctgaacaacaggcagctgagaatgtcaatttcattttgtggaggcatcgggtttgagggaattcacccaaggccactatgggatgaaggttatgaactggatgaccatactgtgagagactgaactagattgggtgtatggctggctgtcactttctgacctaggtcagtctgtactctctgttctcaaggaacagtggcagatgggaggagagctggggcaattctttcagattgtggtttatatgggaaattgttcatggtccccagcctctcaatgtatgtctgtagatgcattgtgaacgcatgctgcagaaggcctttgctcatttgaactgtgtagaggcaagaagtgaccggctcatgtcaatgctgtctacaaacgagtatgatcctaactgttttggataatcttttatatttctgaactctgaatttaatcattttattagattaaaatatgcgat

SEQ ID NO: 9 - protein, 446 aa, Mus musculus
  mouse ortholog of human smooth muscle associated protein-2 (SMAP-2), secreted modular calcium-binding protein-2 (SMOC-2)
MLPPQLCWLPLLAALLPPVPAQKFSALTFLRVDQDKDRDCSLDCPSSPQKPLCASDGRTFLSRCEFQRAKCKDPQLEIAHRGNCKDVSRCVAERKYTQEQARKEFQQVFIPECNDDGTYSQVQCHSYTGYCWCVTPNGRPISGTAVAHKTPRCPGSINEKVPQREGAGKADDAAAPALETQPQGDEEDIASRYPTLWTEQVKSRQNKTNKNSASSCDQEHQSALEEAKQPKNDNVVIPECAHGGLYKPVQCHPSTGYCWCVLVDTGRPIPGTSTRYEQPKCDNTARAHPAKARDLYKNRPLQGCPGAKKHEFLTSVLDALSTDMVHAVSDPSSSSGRLSEPDPSHTLEERVVHWYFKLLDKNSSGDIGKKEIKPFKRFLRKKSKPKKCVKKFVEYCDMNNDKSITVQELMGCLGVTREEGKANTRKRHTPRGNAESTSNRQPRKQG

SEQ ID NO: 10 - DNA, 1235 bp, Homo sapiens
  human 1.2 kb SMAP-2 promoter
ccaatgcccctgacaagtcaattgattcattgttcttgaacaaatcttcactcttctggctttctgtgtgagacagaattagggctttttcctgtaaagttataagccaaggctaaactatgtcagcaatgttgtctgcagtggatattatccagagtctgggtgtgcaaaaatttcctggtttccaacgtagtcttcgtgggtgagtttacaacctccacaagccctgtgctccccgcacaaacacctgaaatgctttgacagagctgtttaaaaacagccacatgtgtctgtgaatgatgcccaaccacatgctcccccgacttgagaggatcaaggctacagcccaggtcagaccccggatctttaggacctaaattcccccatccctgcagtggataaccccgtgtaaagatgccaaaggcgggctgcccccggcgcagcctttcctggacatgtgttcccagctgcctttgctggagcgccgctgatccttcagcacctctgagaggcaatgttctgggctggaccagggggccttagagggcagaggtcaaggagaaccccggagtctggggaccggccaaggcttggcgttaacctttgtggatgaaaagtcccgctgggctgatcctgcgcggaccgggcctggaccctggggcgtgaggagggcgcggtgcgtcccgtggttgtgcttggaagccccccgagggtgcgcgcgcgtgggtatgagtgcgtgcgtgtgcctgggtgtgcgtgtgtgtaagtgtgcacgtgtgtgtgtgagagtgcgcgcggggaaggaggcacagagacagcccggacaggccactgcgcagccctggtggcccccgctccacctctcgctccgcagacccgcgccagggaggcctctgggccgcagcgggcaccggagcggagcgggcgcggcagcgggcgctgggaggtggggctgggggaggagagggggagggagagaggcgggcgggaggggaggatccgggaagctccggggtatttgacaggagcgagggcggacgcaaagaacgcggaggacctctgggtgcctgcaggggagctgctccagccgggccgccgggagcggtggggagagcatcgcggagccgcccctccacgcgcccgcccagccgcgctcgcccactgggctctcccggctgcagtgccagggcgcaggacgcggccgatctcccgctcccgccacctccgccaccatg

SEQ ID NO: 11 - DNA, 986 bp, Mus sp.
  mouse SMAP-2 promoter
tgttttgctccctacagagaggccctgacaagtcaattgattctttgttctcgaaagtcttcccagctggctttctgcgtgaggtagaattagggccttttcctgtgaagctaccagccagggctaaactatttcagcaatgctccttttgcaggacctggtgcgcaaaatccccttggtttctatcacagtcaaaagcctactcaaatgcgcttgctgccgaacacctgaaatgcttgcctaaactgtttgaagcggccacatcaatgtgtgcgctcacctttctctaagtttgcccaactcaaggaagtcatctgcctcacagatctgttttcagacccctcccgccctcctcgttaacctgcggaaagatgcaggactcctggtgttctcagaggccctaccgaggagccaatgaccacttagcatctcagagtgctacattctggtgaatgtaaagccacaggcgagccaaggcacagtggggaccctccaaggtgaagacagaaggcgccctaggccaggagggtggggctcagtgcatctcttggagccagtgtgagtgtgcatgcctgtaaggtgtgccctggcattggcttgaaggaggcgtgcaaacagcatcaacacacgcttgcctagtcctggtagccttcgctgtctccttcacagccctgcgcgccgggcggcccctgcgcgggcgcggtggctaagggaggtggggcggagggaggagatttgcgggcgggagggcgaagagggcgggacaggaggatccggggaaactagggcgggttgacagcagcagcggcagttcggggagcgccggccagagcgcacggaggggccgtgcggtctccaccagcggccataggaccgcggagcagttctagcccgctcgcccgcacgttcgcacaccggatcttcgccgagtgccagggcgcagcgcgtggggcgtctgcctcgcttggtcccctccagcgtcacca

SEQ ID NO: 12 - protein, 4 aa, Artificial Sequence
  Description of Artificial SequenceSMAP-2 thyroglobulin-1 repeat domain central core conserved residues
CWCV

SEQ ID NO: 13 - protein, 6 aa, Artificial Sequence
  Description of Artificial Sequence hexahistidine affinity tag (His)
HHHHHH

SEQ ID NO: 14 - protein, 200 aa, Artificial Sequence
  Description of Artificial Sequencepoly-Gly flexible linker
GGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGG

SEQ ID NO: 15 - DNA, 25 bp, Artificial Sequence
  Description of Artificial Sequencehuman SMAP-2 Taqman probe
ngaccaacagaactggtctgcgtgc

SEQ ID NO: 16 - DNA, 19 bp, Artificial Sequence
  Description of Artificial Sequencehuman SMAP-2 forward quantitative PCR primer
ggagtggaatgcccacaca

SEQ ID NO: 17 - DNA, 19 bp, Artificial Sequence
  Description of Artificial Sequencehuman SMAP-2 reverse quantitative PCR primer
tgagggtggcagctggtta

SEQ ID NO: 18 - DNA, 21 bp, Artificial Sequence
  Description of Artificial Sequencemouse SMOC-2 forward PCR primer
tgggtcacacaggcactagct

SEQ ID NO: 19 - DNA, 24 bp, Artificial Sequence
  Description of Artificial Sequencemouse SMOC-2 reverse PCR primer
tggagcctaacagcatagtctcaa

SEQ ID NO: 20 - protein, 446 aa, Artificial Sequence
  Description of Artificial Sequenceconsensus sequence of human SMAP2 short, SMAP2 long and mouse SMAP2
MLLPQLCWLPLLAGLLPPVPAQKFSALTFLRVDQDKDKDCSLDCAGSPQKPLCASDGRTFLSRCEFQRAKCKDPQLEIAYRGNCKDVSRCVAERKYTQEQARKEFQQVFIPECNDDGTYSQVQCHSYTGYCWCVTPNGRPISGTAVAHKTPRCPGSVNEKLPQREGTGKTDDAAAPALETQPQGDEEDIASRYPTLWTEQVKSRQNKTNKNSVSSCDQEHQSALEEAKQPKNDNVVIPECAHGGLYKPVQCHPSTGYCWCVLVDTGRPIPGTSTRYEQPKCDNTARAHPAKARDLYKGRQLQGCPGAKKHEFLTSVLDALSTDMVHAASDPSSSSGRLSEPDPSHTLEERVVHWYFKLLDKNSSGDIGKKEIKPFKRFLRKKSKPKKCVKKFVEYCDVNNDKSISVQELMGCLGVAKEDGKADTKKRHTPRGHAESTSNRQPRKQG